(19) United States
(12) Patent Application Publication
(10) Pub. No.: US 2021/0403571 A1
(43) Pub. Date: Dec. 30, 2021

(54) SYNERGISTIC COMBINATIONS OF METHIONINE DEPLETION AGENTS AND IMMUNE CHECKPOINT MODULATORS

(71) Applicant: ERYTECH PHARMA, LYON (FR)

(72) Inventors: Vanessa BOURGEAUX, L'ISLE D'ABEAU (FR); Alexander SCHEER, CASTRES (FR); Karine SENECHAL, GENAS (FR)

(21) Appl. No.: 17/293,708

(22) PCT Filed: Nov. 14, 2019
(86) PCT No.: PCT/EP2019/081388

§ 371 Date: May 13, 2021

Related U.S. Application Data
(60) Provisional application No. 62/768,036, filed on Nov. 15, 2018.
(60) Provisional application No. 62/824,249, filed on Mar. 26, 2019.

(51) Int. Cl. C07K 16/28; A61K 45/06; A61P 35/00

(52) U.S. Cl. CPC C07K 16/2827; C07K 16/2818; A61K 2039/505; A61P 35/00; A61K 45/06

(57) ABSTRACT

The invention concerns a pharmaceutical composition, kit or fixed-dose combination comprising a methionine depletion agent (MDA), and an anti-cancer immune modulator (ACIM), for use in the treatment of a disease or condition in a subject or patient in need of treatment thereof. Synergic combinations are provided. Cancer may be for example acute lymphoblastic leukemia (ALL), acute myeloid leukemia (AML), pancreatic cancer, gastric cancer, colorectal cancer, prostate cancer, ovarian cancer, brain cancer, head and neck cancer or breast cancer.

### CROSS-REFERENCE TO RELATED APPLICATIONS AND INCORPORATION BY REFERENCE

This application claims the benefit of U.S. Provisional Patent Application No. 62/768,036, filed Nov. 15, 2018, and U.S. Provisional Patent Application No. 62/824,249, filed Mar. 26, 2019, each of which are incorporated by reference in their entirety.

### INCORPORATION BY REFERENCE OF SEQUENCE LISTING PROVIDED AS A TEXT FILE

A Sequence Listing is provided herewith as a text file, “ERY2018002_SeqList_ST25.txt” created on Nov. 13, 2019 and having a size of 8 KB. The contents of the text file are incorporated by reference herein in their entirety.

### FIELD OF THE INVENTION

The present disclosure relates to the use of methionine (MET) depletion agents including ERY-MET™ (methionine gamma lyase) and diets low in MET, in combination with cancer immunotherapies including immune checkpoint modulators (ICM), for treating various cancers, especially those that have become resistant to ICM therapies, including α-PD-1 antibody therapies.

### SUMMARY OF THE INVENTION

Although anti-PD-1 immunotherapy has demonstrated efficacy against several types of cancer, the emergence of resistance mechanisms has limited its use (O'Donnell 2016, Gong 2018). For example, the overexpression of adenosine receptors (A_{2A}R) on the surface of infiltrated CD8⁺ T cells following anti-PD-1 treatment attenuates the anti-tumor immune response in an adenosine-rich tumor microenvironment (TME) (Mittal 2014; Allard 2013). In addition, there is evidence that the hypermethylation of certain genetic loci (e.g. PD-L1 promoter methylation) contributes to such immune escape (Serrano 2011; Zhang 2017). Accordingly, as suggested by the diagram 1 placed at the end of the description, one potential way to restore sensitivity to immunotherapy could be to reduce methionine levels (i.e. to reduce both SAM and adenosine levels). That being said, the evidence is incomplete (and/or inconsistent) as regards the impact of systemic methionine depletion on methylation-dependent biological and pathological processes (As4Sanderson, 2019). Moreover, it is not known whether the levels of methionine required to sufficiently reduce adenosine levels in the tumor microenvironment would bu7e safe for a subject or patient. Still, one group has shown that dietary methionine restriction (MR) may promote the differentiation of antitumor macrophages, and, that protein restriction may play a supportive role for immunotherapies (Orillion 2018). The foregoing notwithstanding, there remains a need to develop safe and effective therapies to relieve immune suppression, including that brought on by immunotherapies.

Erymethionase (ERY-MET™, or methionine-gamma-lyase encapsulated into red blood cells) is an innovative therapeutic product capable of reducing systemic levels of L-methionine (U.S. Pat. No. 10,046,009 B2 and WO 2017/114966 A1, both to Erytech Pharma, and herein incorporated by reference in their entireties). Importantly, ERY-MET™ has demonstrated safety and efficacy in several mouse models of cancer, including glioblastoma (Gay F. et al., Cancer Med., 2017 June; 6(6):1437-1452) and gastric cancer (Bourgeaux V. et al., J. Clin. Oncology, 2017, Abstract 78). However, until this disclosure, it was unknown whether ERY-MET™ could be combined safely and effectively with immune checkpoint modulators (ICM), including anti-PD-1 antibodies.

In view of the foregoing, Applicants hypothesized that treatment with ERY-MET™ would both reduce hypermethylation and indirectly decrease adenosine levels in the tumor microenvironment (TME), with the ultimate effect of enhanced activation of silenced/inactivated T cells. Applicants further hypothesized that this potential bimodal action of ERY-MET™ could make it a promising agent to combine with one or more immune checkpoint inhibitor. The benefit of such a combination (e.g. ERY-MET™ and a PD-1 blocking agent) was therefore investigated in a TNBC mouse model.

The effect of drug combination is inherently unpredictable. For example, one drug may partially or completely inhibit the effect(s) of the other. In vivo studies were carried out to assess the ability of combinations of ERY-MET™ to overcome the resistance effects seen by treatment with PD-1 blocking agents. A mouse model of TN BC was used to evaluate the safety and efficacy of various combinations of ERY-MET™ and α-PD-1 antibodies. All treatments were well tolerated and the highest dose of ERY-MET™+α-PD-1 showed a significant growth inhibition at D20 and D23 and an increase in survival of animals. To Applicants knowledge, this is the first in vivo demonstration of α-PD-1 therapy potentiation using a methionine depletion agent (MDA). FIGS. 1 to 4 summarize these data, which collectively lend support to the assertion that the inventive combinations can overcome resistance to α-PD-1 therapy.

In view of these surprising and unexpected results, a first object of the disclosure is to provide therapeutically effective combinations of methionine (MET) depletion agents (“MDA”) including ERY-MET™ (methionine gamma lyase, or “MGL”, encapsulated in erythroid cells), in combination with cancer immunotherapies including immune checkpoint modulators (ICM), particularly including immune checkpoint inhibitors (ICI). MDA also includes, but not solely: any methioninase (METase); a METase as disclosed in WO 2017/114966 A1 (to Erytech), U.S. Pat. No. 9,051,562 (to INSERM et al.), U.S. Pat. No. 8,709,407 (to University of Texas) or U.S. Pat. No. 9,816,083 (to Guangzhou Sinogen); and fumagillin.

In some embodiments, the MDA is ERY-MET™ and the ICM is an ICI such as an α-PD-1 antibody. For examples, the ICI may comprise any or combinations of the following: Nivolumab (OPDIVO®), Pembrolizumab (KEYTRUDA®), BGB-A317, Atezolizumab, Avelumab, Durvalumab, and Ipilimumab (YERVOY®). Now that the disclosure has been made, the skilled artisan will reasonably expect that a safe and effective amount of any PD-1 blocking approach will synergize with the MDA to kill tumor cells, including solid tumor cells, including TNBC tumor cells. As further disclosed below, the efficacy of the combination of the MDA and the ICI is greater than the additive efficacy of either component by itself. Inventors envision that other combinations of MDA and ICI will also yield synergistic efficacy against cells from various cancers.

In some embodiments, the therapeutically effective combinations provide synergistic efficacy against one or more cancers as compared with the efficacy of either the MDA or the ICI alone.

In still other embodiments, the combination is therapeutically effective against cancer types that neither or only one of the MDA or the ICI demonstrate therapeutic efficacy.

By “deprivation”, it is meant a sufficient reduction of methionine to produce beneficial effects in treating cancer, the cancer cells being deprived for sufficient amount of the amino acid.

By “enzyme treatment”, it is meant that the enzyme will degrade the concerned amino acid and possibly induce other beneficial effects such as inhibition of protein or amino acid synthesis or any mechanism that leads to lack of sufficient amount of the amino acid to the cancer cell.

In some particular embodiments, the MDA is a METase and the ICI is a PD-1 blocking agent, including an α-PD-1 antibody, each active ingredient present in amounts that would be subtherapeutic were they to be administered as monotherapies. As used herein, a “subtherapeutic amount” means an amount of a drug or therapeutic agent that is ineffective at producing or eliciting a given therapeutic effect (e.g. a significant reduction in the size of a tumor, a significant decrease in the number of tumor cells or a significant decrease in the metastatic potential of tumor cells).

In a second object, the disclosure provides methods of treating diseases including cancers comprising sequential or simultaneous administration of synergistically effective combinations of MDA and ICI as disclosed herein.

In the context of the invention under its different aspects or objects, at least one “sequential administration” means that the same mammal may be treated sequentially more than once during a treatment therapy or phase. However, one or several methioninase administration(s) may be performed before, during or after one or several PD-1 blocking agent administration(s). In general, if the medicaments are administered at about the same time, the term “simultaneous administration” applies.

In a third object, the disclosure provides kits comprising effective amounts of an MDA and an ICI, optionally including instructions for use thereof in treating cancers.

In a fourth object, the disclosure provides methods of manufacture of a medicament comprising effective amounts of an MDA and an ICI.

In a fifth object, the disclosure provides methods and/or uses of combinations of MDA and ICI in the treatment of cancer. In some embodiments, the use is effective in inducing tumor cells that are resistant to treatment with either the MDA or the ICI alone. In some embodiments, the use of the combination of MDA and ICI is effective in treating a patient in whom a cancer has relapsed after a treatment with either the MDA or ICI previously administered as a monotherapy, or in combination with an agent other than the MDA (in the case where the ICI was previously administered) or the ICI (in the case where the MDA was previously administered).

In a sixth object, the disclosure provides methods and/or uses of combinations of MDA and ICI in the treatment of cancer that is resistant to either or both of the MDA or the ICI, when administered alone or with an agent other than the corresponding MDA or ICI. In some embodiments, simultaneous or sequential administration of individually subtherapeutic doses of the MDA and ICI restores the sensitivity of the tumor cells. In some embodiments, the entire population of tumor cells is killed by a combination of the MDA and ICI, but not either the MDA or ICI alone.

Another object of the present invention is the use of methioninase and a PD-1 blocking agent for the preparation of a pharmaceutical composition or pharmaceutical compositions or a kit or set of pharmaceutical compositions (one containing methioninase, another one containing anti-PD-1), wherein the composition(s) or the kit is for use in treating cancer in a mammal with at least one sequential or simultaneous administration.

Other objects of the invention are:
a pharmaceutical composition comprising a PD-1 blocking agent for use in treating cancer in a mammal, wherein the composition is to be administered to a mammal that has been administered methioninase;
a pharmaceutical composition comprising a PD-1 blocking agent for use in treating cancer in a mammal, wherein the composition is to be administered to a mammal that has been subjected to methionine deprivation diet, i.e. has been administered a methionine deprived food, therapeutic or not; by therapeutic food in the meaning of this invention, it is meant a food administered in medical environment and/or subjected to marketing authorization by Regulatory Authority, especially a liquid food, that may be or not administered by infusion;
a pharmaceutical composition comprising methioninase for use in treating cancer in a mammal, wherein the composition is to be administered to a mammal that will be further administered a PD-1 blocking agent;
a food composition or diet, therapeutic or not, comprising no methionine or substantially no methionine for use in depriving a mammal for methionine, before, during or after treating the mammal with PD-1 blocking agent.

Other objects of the invention include:
the use of a PD-1 blocking agent for the preparation of a pharmaceutical composition for use in treating cancer in a mammal, wherein the composition is to be administered to a mammal that has been administered methioninase;
the use of a PD-1 blocking agent for the preparation of a pharmaceutical composition for use in treating cancer in a mammal, wherein the composition is to be administered to a mammal that has been subjected to methionine deprivation diet, i.e. has been administered a methionine deprived food, therapeutic or not;
the use of a PD-1 blocking agent for the preparation of a pharmaceutical composition for use in treating cancer in a mammal, wherein the composition is to be administered to a mammal that will be further administered methioninase.

Still another object of the invention is a kit comprising a pharmaceutical composition containing methioninase or a therapeutic food or diet for methionine deprivation, and a pharmaceutical composition containing a PD-1 blocking agent, the compositions being separately or jointly packaged. The compositions are for simultaneous or sequential administration with methioninase or food/diet being administered before, after or during the PD-1 blocking agent. The kit may further contain a leaflet indicating that the compositions are for simultaneous or sequential administration with methioninase or food/diet being administered before, during or after the PD-1 blocking agent.

Still another object of the invention is a method of treatment of cancer in a mammal comprising administering to a mammal first an effective amount of methioninase and second an effective amount of PD-1 blocking agent.

Still another object of the invention is a method of treatment of cancer in a mammal comprising administering to a mammal first a food or diet, therapeutic or not, to deprive methionine, and second an effective amount of a PD-1 blocking agent.

Still another object of the invention is a method of treatment of cancer in a mammal having a low methionine bioavailable level, or having been subjected to a food or diet, therapeutic or not, having deprived methionine, the method comprising administering to the mammal an effective amount of PD-1 blocking agent.

In these different objects, methioninase administration and methionine diet deprivation may be combined. Methionine dietary depletion may also be accomplished via orally supplied methioninase activity. For example, some dosage forms containing enzymes may be taken orally with retained enzyme activity in the small intestines. Administration of such preparations would effectively reduce the dietary intake of methionine. In other embodiments, probiotic bacteria harboring methioninase may be administered to patients for whom reduced levels of methionine are desired (see Isabella et al. 2018).

The invention may be beneficial to any cancer, including liquid, i.e. hematological cancers, lymphomas and solid cancers.

A specific object of the invention is the application of this invention to the treatment of cancers auxotrophic or not auxotrophic to methionine and/or ones that when treated with a methionine depletion agent (MDA) respond more robustly to treatment with a PD-1 blocking agent. In advantageous embodiments, cancers that have become resistant to PD-1 blocking agents once more responsive to the PD-1 blocking agents as a result of the treatment with the MDA.

It is a further object of the invention to not encompass within the invention any previously known product, process of making the product, or method of using the product such that the Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

These and other embodiments are disclosed or are obvious from and encompassed by, the following Detailed Description.

### BRIEF DESCRIPTION OF THE DRAWINGS

FIG. 1 is a graph showing the mean tumor volumes (mm³) for mice in Groups G1 to G7 at various days post-tumor implantation;

FIG. 2 is a graph showing the percent of mice surviving at the indicated days post-tumor implantation;

FIG. 3 is a graph showing individual tumor growth for the mice in G3 and G6;

FIG. 4 is a graph showing individual tumor growth for the mice in G3 and G7;

FIG. 5 is a graph showing PD-L1 expression on EMT6 tumor cells at 48 h following the indicated treatments;

FIG. 6 presents graphs showing the effect of MGL alone or in combination with anti-PD-1 (nivolumab) on IFN-γ production in a Mo-DC:T cell MLR;

FIG. 7 is a graph showing the effect of MGL alone or in combination with anti-PD-L1 (atezolizumab) on IFN-γ production in a Mo-DC:T cell MLR;

FIG. 8 presents graphs showing the effect of MGL alone or in combination with anti-CTLA-4 (ipilimumab) on IFN-γ production in a PBMC:PBMC MLR;

FIG. 9 presents graphs showing urea cycle metabolites present in Example 1 tumor and plasma samples (untreated, processed RBC vehicle or 60 U/kg ERY-MET™);

FIG. 10 presents graphs showing RedOx status (GSH:GSSG & NAD/NADH) in Example 1 EMT6 tumor samples (untreated, processed RBC vehicle or 60 U/kg ERY-MET™);

FIG. 11 presents graphs showing the methionine, cystathionine and cysteine concentrations in Example 1 plasma samples (untreated, processed RBC vehicle or 60 U/kg ERY-MET™);

FIG. 12 presents graphs showing the 3-hydroxybutyric acid and 2-hydroxybutyric acid concentrations in Example 1 tumor and plasma samples (first page); and graphs showing acetyl CoA and HMG-CoA concentrations in Example 1 tumor samples, and the acetoacetic acid concentrations in plasma samples;

FIG. 13 presents graphs showing lactic acid concentrations in Example 1 tumor samples;

FIG. 14 presents graphs showing 4-acetamidobutanoic acid, fumarate and malic acid concentrations in Example 1 tumor and plasma samples;

FIG. 15 is a graph showing the concentration of alanine (a ketogenic amino acid) in the plasma samples of Example 1.

### DETAILED DESCRIPTION OF THE INVENTION

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

As discussed above, acquired resistance to anti-PD-1 therapy have urged researchers to combine other anti-cancer agents with α-PD-1 antibodies. Applicants hypothesized that such resistance could be overcome by treating cancer patients with effective combinations of methionine depletion agents (“MDA”) including as ERY-MET™ with ICI including α-PD-1.

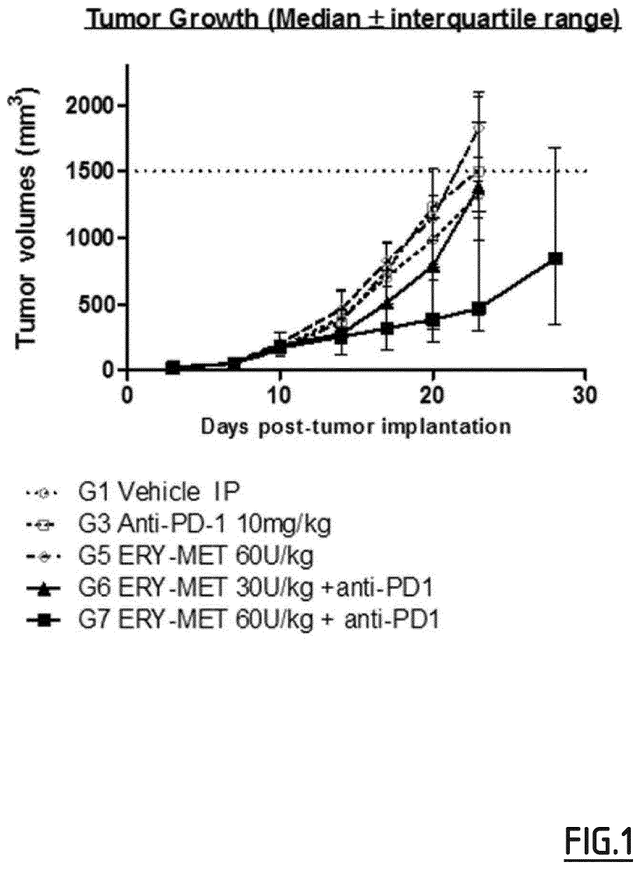

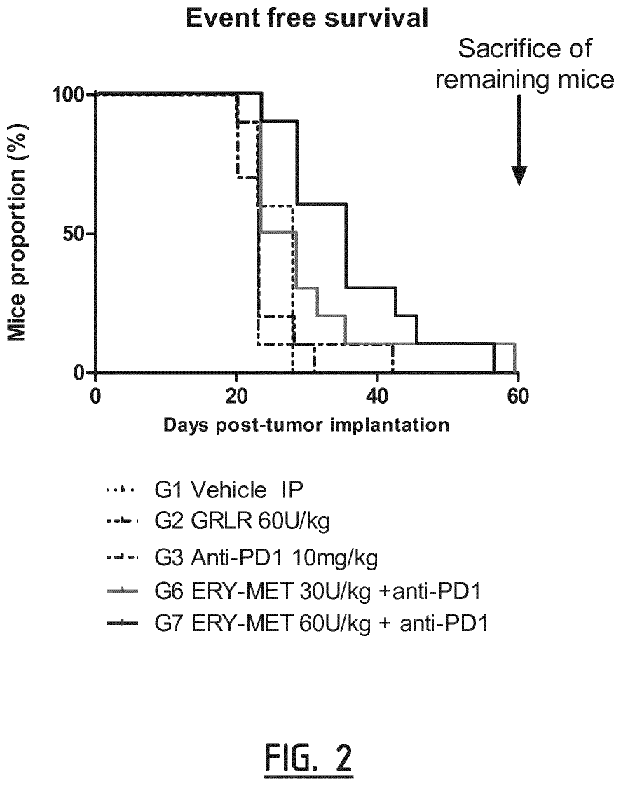

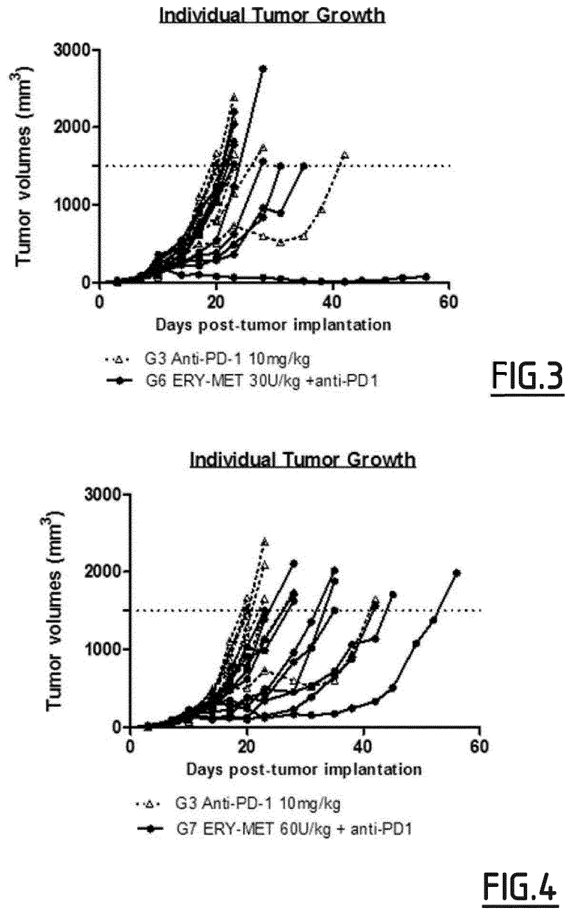

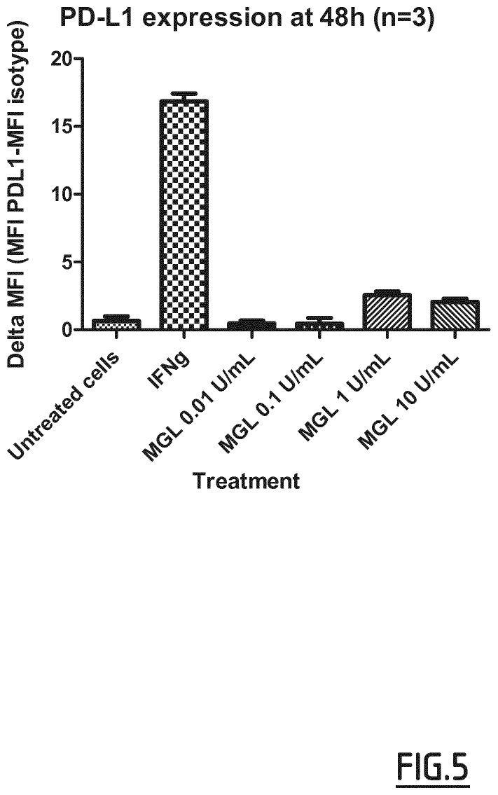

As detailed in the Examples below, mice bearing breast carcinoma were intravenously injected once weekly for 4 consecutive weeks with mouse ERY-MET™ at 30 U/kg or 60 U/kg alone or in combination with α-PD-1 antibody (intraperitoneal, 10 mg/kg, twice weekly for 3 consecutive weeks) from D7 (D0 referring to injection of tumor cells). The average tumor volume was approximately 80 mm³ at the time of the first treatment(s), as is typical for mouse studies evaluating the impact of α-PD-1 antibodies. Moreover, ERY-MET™ treatment was accompanied by daily oral administration of PN (precursor to the MGL co-factor PLP; see Erytech's U.S. Pat. No. 10,046,009 B2). All treatments were well tolerated and the highest dose of ERY-MET™+α-PD-1 showed a significant growth inhibition at D20 and D23 and an increase in survival of animals (FIG. 1). To Applicants knowledge, this is the first in vivo demonstration of such a substantial α-PD-1 therapy potentiation using an enzyme-based MDA. FIGS. 1-4 are graphs showing the impact of the various treatments on tumor growth and event-free survival (EFS) and FIG. 5 shows the impact of increasing concentrations of MGL on PD-L1 expression.

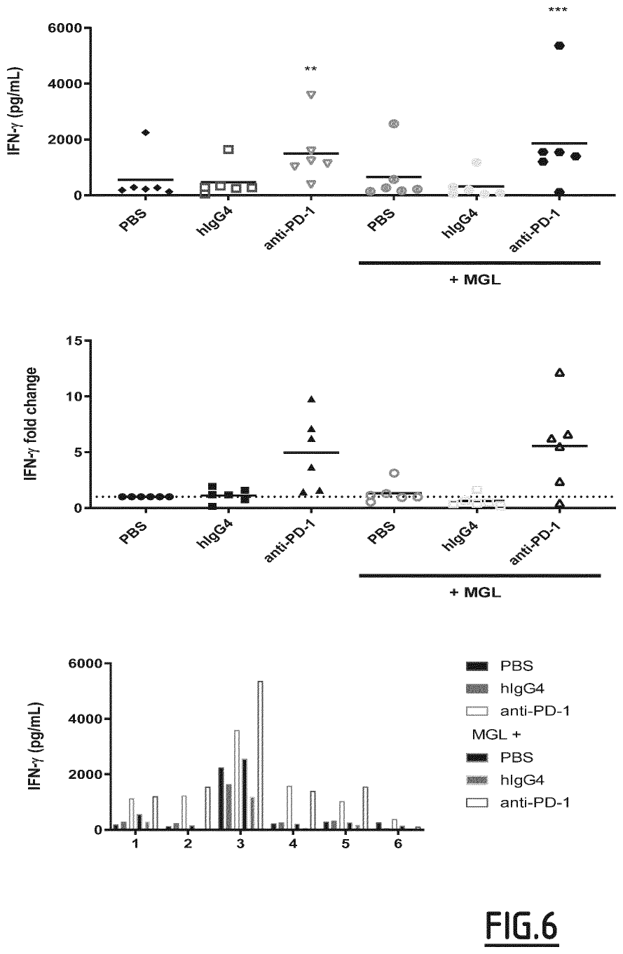

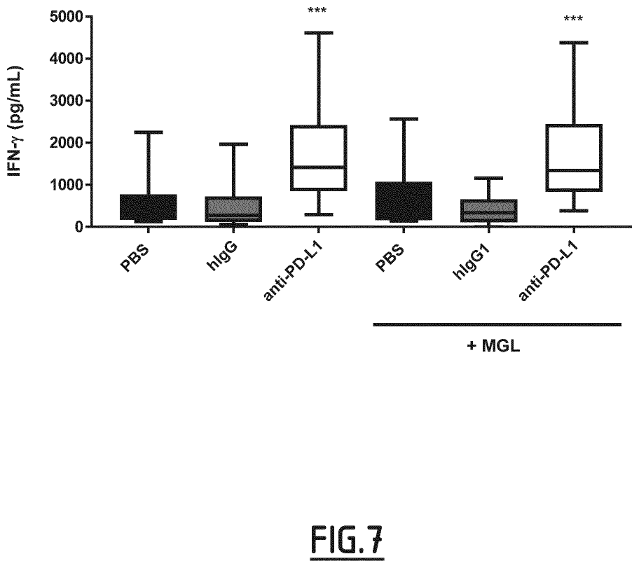

To more completely understand how ERY-MET™ potentiated (or even rescued) the anti-tumor efficacy of immune checkpoint inhibitors (e.g. α-PD-1 antibodies), Applicants measured a variety of markers, including cytokines, metabolites and other analytes, both from the plasma and from the tumors themselves. Importantly, measurements from “tumors” necessarily reflected the conditions of a combination of both the intracellular and extracellular tumor compartments. In contrast, measurements from “plasma” primarily reflected the conditions of the extracellular compartment. FIGS. 6-15 present these data, and an ongoing analysis of collected tumors will allow for the validation of mechanism(s) of action (MOA) proposed herein.

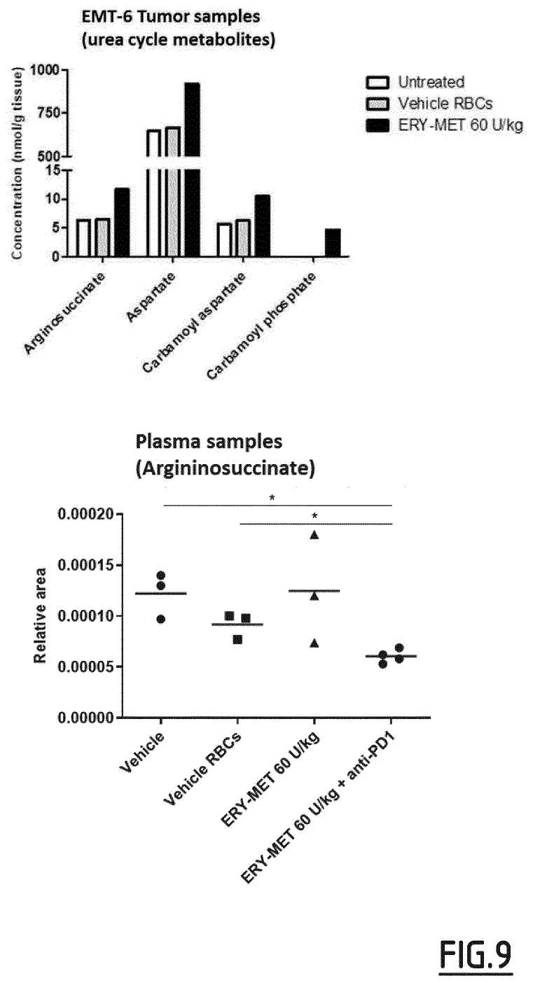

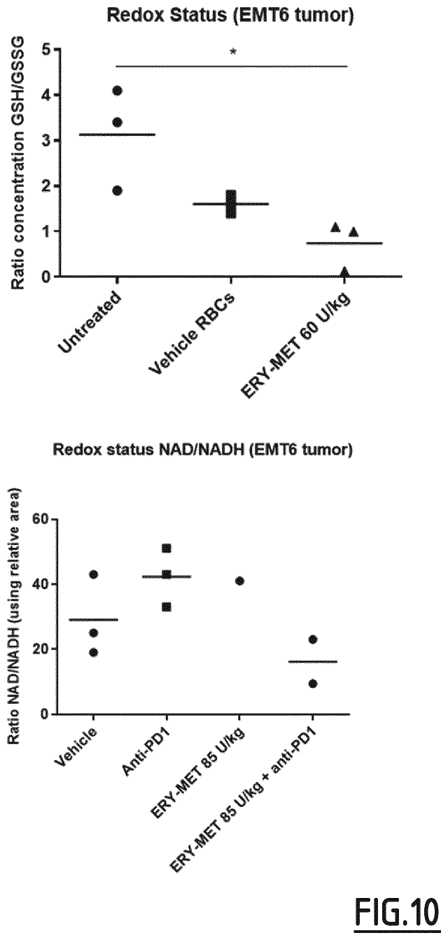

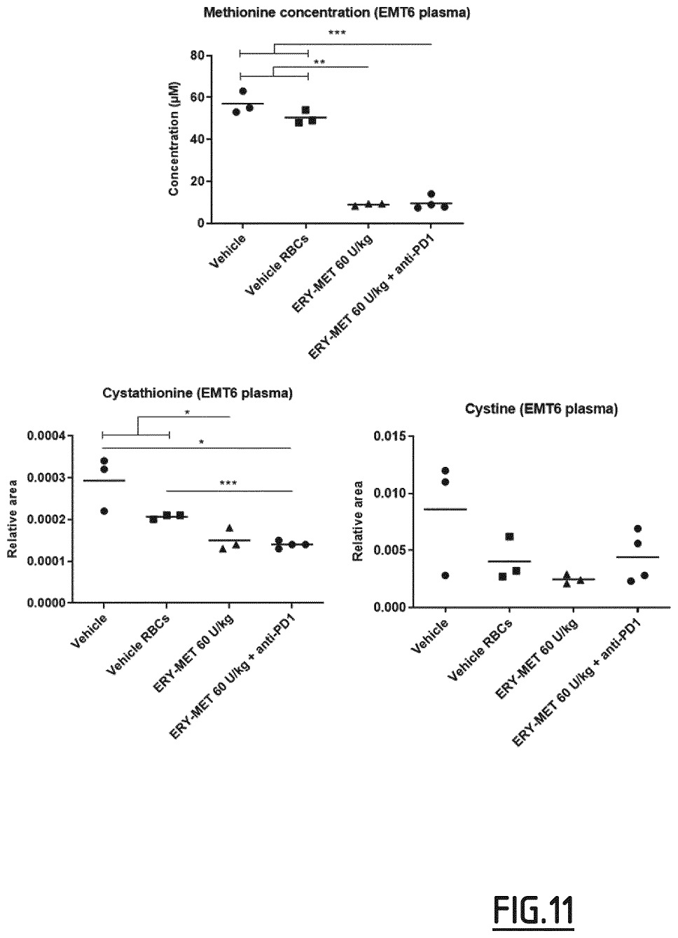

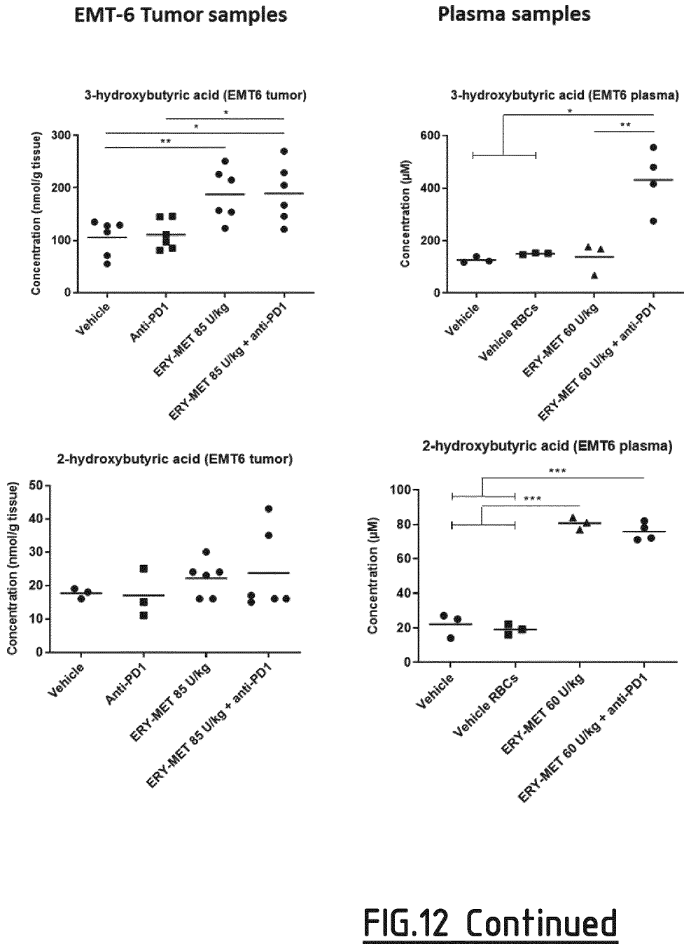

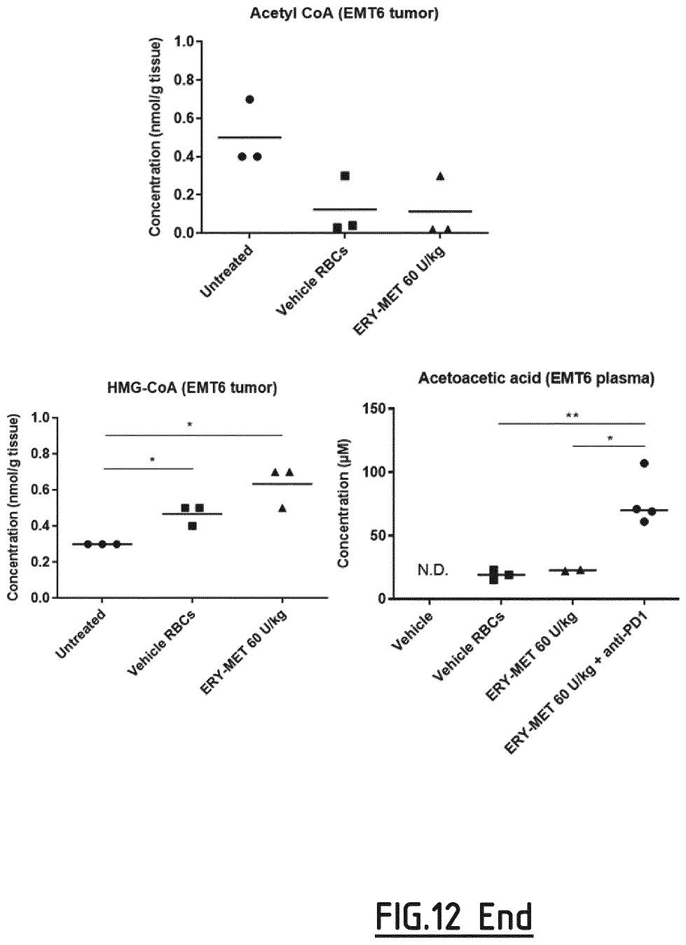

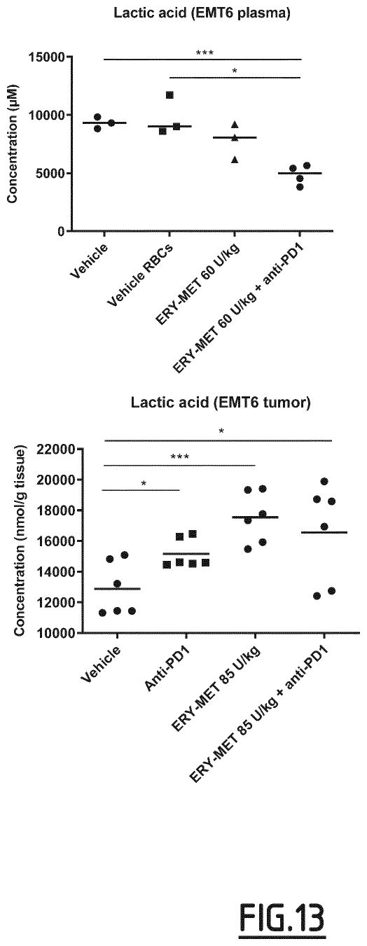

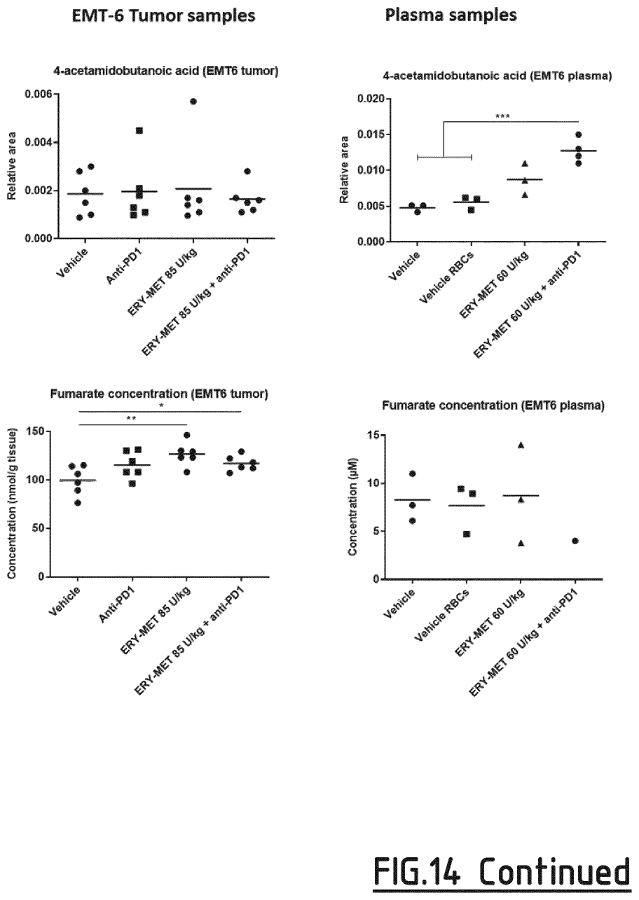

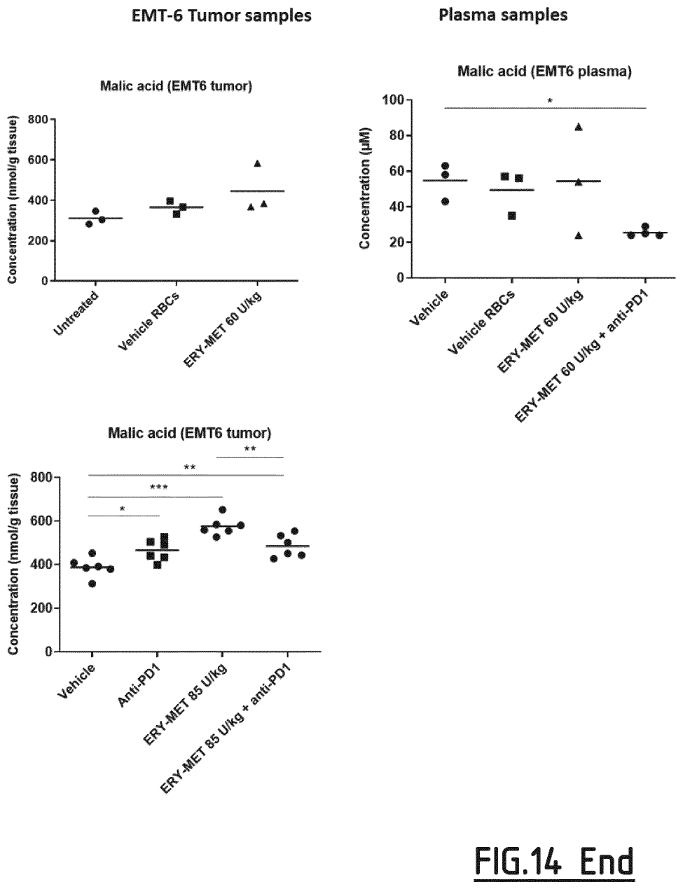

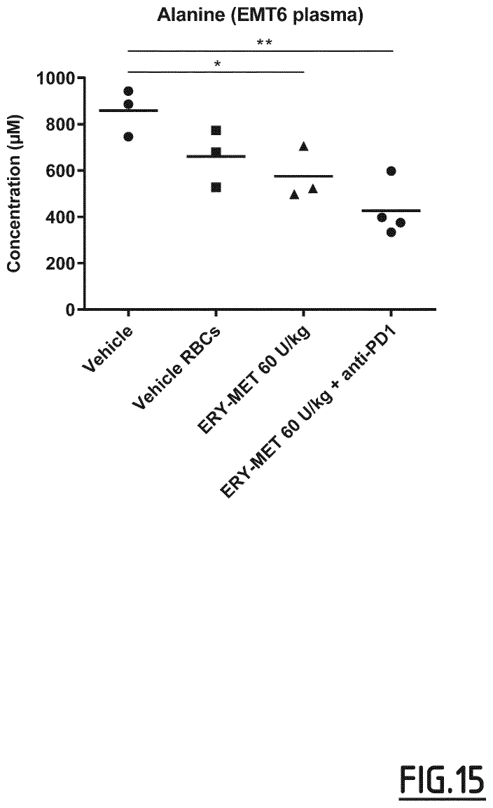

For example, some of the data indicate that the MOA may comprise one or more of the following:
Methionine depleting agents (MDA) may sensitize tumor cells to α-PD-1 therapy—at least in part—by increasing PD-L1 expression levels (FIG. 5)
ERY-MET™ may increase plasma argininosuccinate over vehicle RBCs (FIG. 9)
Addition of α-PD-1 Abs to ERY-MET™ may reduce plasma argininosuccinate (FIG. 9)
ERY-MET™ may decrease the ratio of GSH to GSSG in the tumor (FIG. 10)
ERY-MET™ decreases plasma methionine, and this effect is not significantly changed by the addition of α-PD-1 Abs (FIG. 11, top graph)
ERY-MET™ decreases plasma cystathionine (precursor to cysteine, which is a dimer of two cysteines), and this effect is not significantly changed by the addition of α-PD-1 Abs (FIG. 11, bottom graphs)
ERY-MET™ increases tumor (but not plasma) 3-hydroxybutyric acid (3HB), and the addition of α-PD-1 Abs appears to have no effect on the level of 3HB in the tumor, but does appear to increase the level of 3HB in the plasma (FIG. 12, top graphs)
Neither ERY-MET™ nor α-PD-1 Abs appear to significantly impact 2-hydroxybutyric acid (2HB) levels in the tumor, and only ERY-MET™ appears to increase 2HB levels in the plasma (FIG. 12 bottom graphs)
ERY-MET™ increases tumor HMG-CoA levels (FIG. 12, second page)
ERY-MET™ does not significantly affect plasma acetoacetic acid levels, whereas α-PD-1 Abs appear to significantly elevate plasma acetoacetic acid levels (FIG. 12, second page)
α-PD-1 Abs decrease plasma lactic acid levels (FIG. 13)
Both ERY-MET™ and α-PD-1 Abs appear to elevate lactic acid levels in the tumor (FIG. 13)
Both ERY-MET™ and α-PD-1 Abs appear to elevate acetamidobutanoic acid levels in the plasma (not in the tumor) (FIG. 14, top graphs)
Both ERY-MET™ and α-PD-1 Abs appear to elevate fumarate levels in the tumor (not in the plasma), but this effect does not appear to be additive (FIG. 14, top graphs)
Both ERY-MET™ and α-PD-1 Abs appear to elevate malic acid levels in the tumor, with α-PD-1 Abs appearing to reduce malic acid levels in the plasma (FIG. 14, second page)
The combination of ERY-MET™ and α-PD-1 Abs significantly lowered plasma alanine levels vs. vehicle (FIG. 15).

Thus, it is an object of this disclosure to provide synergistic combinations of methionine depletion agents (MDA, e.g. METase, and more specifically ERY-MET™) and PD-1 blocking agents (e.g. ICI including α-PD-1 antibody) for use in treating patients in need thereof. Other ICI include but are not limited to the following: Ipilimumab (CTLA-4), Nivolumab (PD-1), Pembrolizumab (PD-1), Atezolizumab (PD-L1), Avelumab (PD-L1), Durvalumab (PD-L1), an affimer biotherapeutic inhibitor (PD-L1) (AVACTA), biosimilars thereof and combinations thereof.

It is a further object to provide use of the foregoing combinations to practice methods of treating a subject or patient suffering from cancer comprising simultaneously or sequentially administering synergistically effective amounts of an MDA (e.g. METase or ERY-MET™) and an ICI (e.g. α-PD-1 blocking agent). In some embodiments, the cancer may be a liquid or solid tumor, or a lymphoma. In some embodiments, the use of an MDA may potentiate the solid tumor killing efficacy of otherwise ineffective amounts of ICI. In other embodiments, the ICI may be combined with a better tolerated MDA, such as METase encapsulated in erythrocytes (e.g. Erytech's ERY-MET™). Dietary depletion of methionine may also be used in the practice of the invention.

Determination of a synergistic interaction between an MDA and an ICI may be based on the results obtained from the assays described herein. The results of these assays may be analyzed using the Chou and Talalay combination method and Dose-Effect Analysis with CalcuSyn software in order to obtain a Combination Index (Chou and Talalay, Trends Pharmacol. Sci. 4:450-454; Chou, T. C. (2006) Pharmacological Reviews 68(3):621-681; Chou and Talalay, 1984, Adv. Enzyme Regul. 22:27-55).

As further detailed in the Examples below, the synergistic MDA and ICI combinations provided by this disclosure have been evaluated, and the data can be analyzed utilizing a standard program for quantifying synergism, additivism, and antagonism among anticancer agents. An exemplary program utilized is described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 to 1.2 indicate additive effects. The combination therapy may provide “synergy” and prove “synergistic”, i.e., the effect achieved when the active ingredients used together is greater than the sum of the effects that results from using the compounds separately. A “synergistic effect” may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The person skilled in the art may understand from the present disclosure that the duration of treatment with diet or one of the drugs, and the delay between methionine deprivation and PD-1 blocking agent treatment, may vary depending on the treatment, on the patient response and importantly on the half-life of the drug or diet effect. There may be a difference depending on the dosage form used in the invention, for example a free enzyme, a pegylated enzyme and erythrocytes encapsulating the enzyme, or else enzyme bound to microcapsules (e.g. made of PLA or PLGA) or liposomes or encapsulated in these structures.

In some embodiments of these different objects, the delay between the end of methioninase administration and the initiation of PD-1 blocking agent administration may be between about 1 h and about 7 days, between about 3 h and about 6 days, or between about 1 day and about 5 days. Methioninase may be, for example, free, pegylated or encapsulated.

In another embodiment, the delay between the end of methioninase administration and the initiation of PD-1 blocking agent administration may be between about 1 h and about 30 days, between about 1 day and about 20 days, between about 1 day and about 10 days.

In particular embodiments, the methioninase may be encapsulated, optionally into erythrocytes, and the PD-1 blocking agent may be under any of pharmaceutically acceptable form.

In still another embodiment, the delay between the end of methionine restriction and the initiation of PD-1 blocking agents administration may be between about 1 h and about 7 days, between about 1 h and about 3 days, or between about 1 h and about 1 day.

### Compositions Comprising Free, Pegylated, Encapsulated or Other Enzyme Forms

The disclosed compositions may be administered to a mammal using standard techniques. Techniques and formulations generally may be found in Remington's Pharmaceutical Sciences, 18.sup.th ed., Mack Publishing Co., Easton, Pa., 1990 (hereby incorporated by reference).

Pharmaceutically acceptable carriers and/or excipients can also be incorporated into a pharmaceutical composition according to the invention to facilitate administration of the particular methioninase or asparaginase. Examples of carriers suitable for use in the practice of the invention include calcium carbonate, calcium phosphate, various sugars including lactose, glucose, or sucrose, or types of starch, cellulose derivatives, gelatin, vegetable oils, polyethylene glycols, and physiologically compatible solvents. Examples of physiologically compatible solvents include sterile solutions of water for injection (WFI), saline solution and dextrose.

Pharmaceutical compositions according to the invention can be administered by different routes, including intravenous (e.g. injection or infusion), intraperitoneal, subcutaneous, intramuscular, oral, topical (transdermal), or transmucosal administration. For systemic administration, oral administration may be used. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g. intramuscular, intravenous (including infusion), intraperitoneal, and subcutaneous injection. For injection, pharmaceutical compositions may be formulated in liquid solutions, preferably in physiologically compatible buffers or solutions, such as saline solution, Hank's solution, or Ringer's solution. In addition, the compounds may be formulated in solid form and redissolved or suspended immediately prior to use. For example, lyophilized forms of the methioninase or asparaginase can be used.

Systemic administration may also be accomplished by transmucosal or transdermal means. For transmucosal or transdermal administration, penetrants appropriate to the barrier to be permeated are used in the formulation. Such penetrants are well known in the art, and include, for example, for transmucosal administration, bile salts, and fusidic acid derivatives.

In addition, detergents may be used to facilitate permeation. Transmucosal administration, for example, may be through nasal sprays, inhalers (for pulmonary delivery), rectal suppositories, or vaginal suppositories. For topical administration, compounds can be formulated into ointments, salves, gels, or creams, as is well known in the art.

The invention encompasses also the use of implanted devices or applied on the mammal to deliver the enzyme, for instance through infusion or another route. In a particular embodiment, the device comprises two chambers or vials, one containing methioninase, the other containing a PD-1 blocking agent. The device has, for each chamber or vial, a tube and the like for delivering the active ingredient into the blood circulation, an electronic or electrical valve or pump, or an actuated piston, that may be controlled by an electronic circuit and a suitable software. The electronic circuit and its software controls the delivery of methioninase and/or PD-1 blocking agent.

Compositions comprising erythrocytes (red blood cells or RBCs) encapsulating the enzyme:

In an embodiment, methioninase is encapsulated inside erythrocytes and the composition comprises a suspension of these erythrocytes in a pharmaceutically acceptable carrier or vehicle.

In an embodiment, methioninase is encapsulated inside erythrocytes and the composition comprises a suspension of these erythrocytes in a pharmaceutically acceptable carrier or vehicle.

In an embodiment, methioninase is in free form or under a pegylated form (PEG-methioninase), in a pharmaceutically acceptable carrier or vehicle.

In an embodiment, methioninase is in free form or under a pegylated form (PEG-methioninase), in a pharmaceutically acceptable carrier or vehicle.

In an embodiment, methioninase is administered in an amount of between about 100 and about 100,000 IU, between about 500 and about 50,000 IU, or between about 500 and about 5,000 IU.

In an embodiment, methioninase is administered once in an amount of between about 500 and about 100,000 IU, between about 1,000 and about 50,000 IU, or between about 5,000 and about 30,000 IU.

In an embodiment, the composition is for use for two or more sequential administrations, particularly 2 or 3.

In an embodiment, the methioninase and the PD-1 blocking agent are used sequentially or simultaneously in accordance with the invention, with the methioninase encapsulated into erythrocytes.

In a particular embodiment, the methioninase may be a PEG-methioninase, or an otherwise modified methioninase.

In an embodiment, methioninase and the PD-1 blocking agent are used sequentially or simultaneously in accordance with the invention, with methioninase encapsulated into erythrocytes and the PD-1 blocking agent in any pharmaceutically acceptable form.

“Encapsulated” means that the enzyme is contained inside the erythrocytes, with the further understanding that a small proportion of the enzyme may remain associated with the cell membrane.

### Dietary Methionine Restriction

Dietary methionine restriction has been proposed either in association with cystemustine therapy in melanoma and glioma (E. Thivat et al., Anticancer Research 2009, 29: 5235-5240) or with FOLFOX as first line therapy of metastatic colorectal cancer (X. Durando et al., Oncology 2010, 78: 205-209). Methionine restriction or deprivation diet is a food regimen or feeding the mammal with a food composition during a sufficient time to induce a full or substantial decrease or elimination of free methionine in the mammal.

The food may be a liquid food that is administered through parenteral route, especially infusion.

Also, methionine deprivation using methioninase aims at inducing a full or substantial decrease or elimination of free methionine in the mammal. Typically, this diet is performed in order to decrease the methionine level of 30 to 100%, typically from 30 to 60% with respect to the mean level in the mammal. Reference may be done to the works by Thivat 2009 and Durando 2010.

Administration of the food may be done for one day or more, for example from one day to seven days. In an embodiment, the food is combined to methioninase treatment, for example the food is administered during the whole or part duration of treatment with methioninase.

### Methioninase

Methioninase may be further called, inter alia, L-methioninase, Methionine Gamma Lyase (“MGL”); one such compound having the EC number 4.4.1.11 and CAS number 42616-25-1. In order to be aware of the methioninase sources which may be used according to the invention, mention may notably be made to the publication El Sayed A, Applied Microbial. Biotechnol. (2010) 86: 445-467.

A recombinant methioninase may be produced in the Escherichia coli bacterium from a gene coding for the enzyme, for example from the Pseudomonas putida bacterium. The thereby obtained enzyme called rMETase may be used under free form or under a modified form, e.g. pegylated form (PEG-rMETase). See X. Sun et al. Cancer Research 2003, 63: 8377-8383. It may also be encapsulated into erythrocytes, the composition or suspension advantageously containing an number of erythrocytes and an amount of encapsulated methioninase that is sufficient to deliver to the patient the desired dose of methioninase. The person skilled in the art may refer to WO 2015/121348 (to Erytech Pharma) for compositions and methods of use.

The methioninase component of the composition may further comprise the cofactor of the enzyme, i.e. PLP, and/or a precursor thereof, which may be a non-phosphate precursor, such as a non-phosphate form of vitamin B6, and/or a phosphate precursor such as pyridoxine phosphate (PNP).

Vitamin B6 exists in different forms, either phosphate or non-phosphate. Pyridoxine phosphate (PNP), pyridoxal phosphate (PLP) and pyridoxamine phosphate (PMP) are the phosphate forms thereof. The corresponding non-phosphate forms are pyridoxine (PN), pyridoxal (PL), and pyridoxamine (PM). The non-phosphate forms of vitamin B6 may cross the erythrocyte membrane, which the phosphate forms can only cross with difficulty.

According to the predominant route, pyridoxine (PN) is transformed inside the erythrocytes into PNP under the effect of PN-kinase, PNP is then transformed into PLP under the effect of PNP-oxidase. The PLP may then be transformed into pyridoxal (PL) under the effect of PLP phosphatase and the PL may leave the erythrocytes. It is easily understood that the provided precursor is able to undergo transformations in the erythrocytes during the preparation method or during the storage of the composition.

By a non-phosphate form of vitamin B6, will be meant here one of the three “vitamers” of vitamin B6 or a mixture of two or three vitamers: PL, PN and PM. The PN form is advantageous. They may also be in the form of a salt.

The methioninase component of the composition may comprise PLP encapsulated in erythrocytes. The PLP may be provided during the encapsulation procedure or be totally or partly obtained in the erythrocytes from its precursor. The PLP either present or formed may be associated with the enzyme. The methioninase component of the composition may therefore comprise the corresponding holoenzyme, for example methioninase-PLP. Under these conditions, the half-life of the active enzyme, as observed for example with the duration of the plasma depletion of its substrate, is considerably increased. The methioninase component of the composition notably gives the possibility of preserving enzymatic activity beyond 24 hours after administration, notably at or beyond 1, 5, 10 or 15 days.

The methioninase component may further comprise PLP or a PLP precursor for simultaneous, separate or sequential administration with the methioninase. In an embodiment, the methioninase is encapsulated inside erythrocytes and further provided is a non-phosphate precursor of PLP for separate or sequential administration.

According to an embodiment, the methionine component comprises (i) a formulation of erythrocytes and a pharmaceutically acceptable vehicle, the erythrocytes encapsulating methioninase, and (ii) a formulation of vitamin B6 in a non-phosphate form, particularly PN, and a pharmaceutically acceptable vehicle. These formulations are for simultaneous, separate or sequential administration, and dedicated to methionine depletion according to the invention.

The methioninase component may notably be in the form of a set or kit, comprising separately these formulations and the PD-1 blocking agent. According to an embodiment, the pharmaceutically acceptable vehicle in the formulation of erythrocytes is a “preservation solution” for erythrocytes, i.e. a solution in which the erythrocytes encapsulating an active ingredient are suspended in their suitable form for being stored while awaiting their injection. A preservation solution advantageously comprises at least one agent promoting preservation of the erythrocytes, notably selected from glucose, dextrose, adenine and mannitol. Possibly, the preservation solution contains inorganic phosphate allowing inhibition of the intra-erythrocyte PLP-phosphatase enzyme.

In an embodiment, methioninase encapsulated inside erythrocytes may be administered at least once or at least twice before the PD-1 blocking agent is administered. Moreover, each methioninase administration may be followed by administration of a solution of non-phosphate precursor of PLP before the PD-1 blocking agent is administered. Alternatively, the PD-1 blocking agent may be administered prior to the administration of the methioninase component of the composition.

In general, MGL activity is expressed in International Units (IU), which corresponds to the amount of MGL required to liberate one micromole of ammonia per minute under the following conditions. In the presence of a sufficient amount of its cofactor PLP, MGL hydrolyzes L-methionine into alpha-ketobutyric acid, forming one molecule of ammonium per molecule of L-methionine: L-methionine+H₂O→methanethiol+NH₄⁺+alpha-ketobutyric acid.

The dosage of MGL activity is performed at 37° C., pH=8.6, in presence of 0.26 μg/ml of MGL, 20 nM of PLP and 25 mM of L-methionine, a commercially available test may be used (e.g. NH₃ kit, Roche diagnostics).

The method consists in measuring the kinetics of ammonium production between 5 min and 10 min of the reaction, when maximum activity (Vmax) of MGL is reached. The measurement of ammonium production is obtained by measuring the variation of optical density at 340 nm due to the oxidation of NADPH to NADP′ by the glutamate dehydrogenase (GLDH) in the presence of ammonium and alpha-ketoglutaric acid, as follows: Alpha-ketoglutaric acid+NH₄⁺+NADPH→L-glutamic acid+NADP⁺+H₂O.

In some embodiments, the combination methioninase+PD-1 blocking agent may further comprise other active ingredients, including other amino acid depletion agents (e.g. ASNase). For example, effective combinations of ASNase and METase are disclosed in WO 2017114966 A1 (to Erytech, and herein incorporated by reference in its entirety). Any ASNase may be used, including the following commercial products: 5000 U MEDAC®, 10000 U MEDAC®, ONCASPAR®.

Accordingly, combinations comprising MDA+ICI+at least one other active ingredient are encompassed by the disclosed invention.

Encapsulation into Erythrocytes

According to an embodiment, the methioninase component comprises erythrocytes encapsulating the enzyme and a pharmaceutically acceptable vehicle. Advantageously, the erythrocytes are taken from a mammal of the same species as the treated subject or patient. When the mammal is a human, the erythrocytes are advantageously human erythrocytes. In an embodiment, the erythrocytes come directly from the subject or patient to be administered the combination of MDA and ICI (i.e. autologous erythrocytes).

According to an embodiment, the pharmaceutically acceptable vehicle is a “preservation solution” for erythrocytes (i.e. a solution in which the erythrocytes encapsulating the enzyme are suspended in their suitable form for being stored while awaiting their injection). A preservation solution advantageously comprises at least one agent that promotes the preservation of the erythrocytes, notably selected from glucose, dextrose, adenine and mannitol.

The preservation solution may be an aqueous solution comprising NaCl, adenine and at least one compound from among glucose, dextrose and mannitol.

The preservation solution may comprise NaCl, adenine and dextrose, preferably an AS3 medium (see D'Amici et al. Blood Transfus. 2012 May; 10(Suppl 2): s46-s54, which is herein incorporated by reference in its entirety).

The preservation solution may comprise NaCl, adenine, glucose and mannitol, advantageously a SAG-Mannitol (SAGM) or ADsol medium.

In particular, the composition or suspension, in a preservation solution, may be characterized by an extracellular hemoglobin (Hb) level maintained at a level equal to or less than 0.5, in particular 0.3, notably 0.2, advantageously 0.15, or even more advantageously 0.1 g/dl at 72 h and preservation at a temperature comprised between about 2 and about 8° C.

In particular, the methioninase component of the composition or suspension, in a preservation solution, may be characterized by an extracellular Hb level maintained at a level equal to or less than 0.5, in particular 0.3, notably 0.2, advantageously 0.15, even more advantageously 0.1 g/dl for a period comprised between about 24 h and about 20 days, notably between about 24 and about 72 h and preservation at a temperature comprised between about 2 and about 8° C. The extracellular Hb level may be measured by the manual reference method described in G. B. Blakney and A. J. Dinwoodie, Clin. Biochem. 8, 96-102, 1975, or by any other suitable manual or automated method.

Moreover, the methioninase component of the composition or suspension, in a preservation solution, may be characterized by a hemolysis rate maintained at equal to or less than 2, notably 1.5, advantageously 1% at 72 h and preservation at a temperature comprised between about 2 and about 8° C. In particular, the hemolysis rate may be maintained at equal to or less than 2, notably 1.5, advantageously 1% for a period comprised between about 24 h and about 20 days, notably between 24 and 72 h and at a temperature comprised between about 2 and about 8° C.

### Methods of Encapsulation

Erythrocytes may be encapsulated with a host of active ingredients using a wide range of technical approaches, including at least the following (and techniques yet to be developed): hypotonic loading (see WO 2006/016247 and WO 2017/114966, both to Erytech; US 2016/0051482 A1 to Erydel; and WO 2013/045885, to St. Georges Hospital Medical School), mechanical/microfluidic loading (see US 2018/0201889 A1, to SQZ; WO 2016/109864 A1, to Indee, Inc.; WO 2019/018497 A1, to Harvard), “soluporation” (see US 2017/0356011 A1, US 2019/0194691 A1, and US 2019/0217315 A1, to Avectas), laser-assisted cell loading (see US20190071695A1, to Cellino Biotech, Inc.), cell-penetrating peptide (CPP), electroporation, transfection and genetic expression (see WO 2016/183482 A1 to Rubius). All of the foregoing references are incorporated herein by reference in their entireties.

When hypotonic loading (also referred to as “lysis-resealing”) is used, erythrocytes are exposed to hypotonic conditions to open pores in their membranes to allow active ingredients to enter the cells. Thereafter, the loaded cells are resealed by exposing them to hypertonic conditions. Three methods are routinely used: hypotonic dialysis, hypotonic preswelling and hypotonic dilution.

In hypotonic dialysis, a suspension of erythrocytes encapsulating the active ingredient (e.g. an enzyme) may be advantageously obtained using the following method:

1—suspending a pellet of erythrocytes in an isotonic solution at a hematocrit level equal to or greater than 65%, cooling between about +1 and about +8° C.;

2—subjecting the erythrocytes to a lysis procedure, at a temperature maintained between about +1 and about +8° C., comprising the passing of the suspension of erythrocytes at a hematocrit level equal or greater than 65% and of a cooled hypotonic lysis solution between about +1 and about +8° C., into a dialysis device (e.g. a coil or a dialysis cartridge);

3—subjecting the erythrocytes to an encapsulation procedure by adding the enzyme to be encapsulated into the suspension before or during lysis, at a temperature maintained between about +1 and about +8° C.; and

4—subjecting the erythrocytes to a resealing procedure conducted in the presence of an isotonic or hypertonic, advantageously hypertonic solution, at a higher temperature, notably comprised between about +30 and about +42° C.

In some embodiments, the lysis-resealing methods described in WO 2006/016247 and WO 2017/114966 (both to Erytech Pharma, and incorporated herein by reference in their entireties).

### Methods of Use

In another aspect, the invention comprises a method for treating cancer in a mammal in need thereof, the method comprising depriving the mammal of a sufficient methionine and administering to the mammal a PD-1 blocking agent. In some embodiments, methionine deprivation may be performed as mentioned above through dietary methionine deprivation and/or methioninase administration.

In another aspect, the invention comprises a method for treating cancer in a mammal in need thereof, the method comprising administering, especially injecting or infusing, to the mammal in need thereof, a composition comprising methioninase and a composition comprising a PD-1 blocking agent.

### REFERENCES

Allard et al. “Targeting CD73 enhances the antitumor activity of anti-PD-1 and anti-CTLA-4 mAbs”, Clin Cancer Res. 2013; 19:5626-35.
Beavis P. A. et al., Oncoimmunology. 2015 May 5; 4(11).
Gong et al. “Development of PD-1 and PD-L1 inhibitors as a form of cancer immunotherapy: a comprehensive review of registration trials and future considerations”, Journal for ImmunoTherapy of Cancer (2018) 6:84; 74:3652-8.
Mittal et al. “Antimetastatic effects of blocking PD-1 and the adenosine A2A receptor”, Cancer Res. 201.
O'Donnell et al. “Acquired resistance to anti-PD1 therapy: checkmate to checkpoint blockade?” Genome Medicine (2016) 8:111.
Sanderson, S. M. et al. “Methionine metabolism in health and cancer: a nexus of diet and precision medicine.” Nat Rev Cancer 19, 625-637 (2019).
Serrano et al. “Role of Gene Methylation in Antitumor Immune Response: Implication for Tumor Progression”, Cancers 2011, 3, 1672-1690.
Zhang et al. “PD-L1 promoter methylation mediates the resistance response to anti-PD-1 therapy in NSCLC patients with EGFR-TKI resistance. Oncotarget (2017) 8:101535-44.

The application will now be described further in the following non-limiting Examples.

### EXAMPLES

Breast cancer is the most common cancer in women with 54,000 new cases diagnosed in France in 2015. Triple-negative breast cancers (TNBCs), a subtype defined by the absence of estrogen and progesterone receptors and the lack of HER2 overexpression (ER-PR-HER2-), tends to be more aggressive than other types. Chemotherapy is the primary established systemic treatment for patients with TNBC in both early and advanced-stages of the disease. The lack of targeted therapies and the poor prognosis of TNBC patients have fostered a major effort to discover safe and effective new therapies.

Recently, a metabolic signature of breast cancer has been identified in patient plasma that suggested an increased utilization of the amino acid methionine (Jove 2017), providing a scientific rationale for the treatment of breast cancer with ERY-MET™. In addition, Applicants hypothesized that by influencing methionine metabolism, ERY-MET™ could also decrease SAM levels and indirectly reduce the concentration of the immunosuppressive adenosine metabolite.

### Example 1—Erymethionase/ERY-MET™ (Methionine-Gamma-Lyase-Encapsulated into Red Blood Cells) Potentiates Anti-PD1 Therapy in EMT-6 TNBC Syngeneic Mouse Model

Study Aim. To evaluate the antitumor activity of ERY-MET™ (Erytech's erythrocyte encapsulated MGL)/PN (orally available vitamin B6 sold as BECILAN®, by DB Pharma, as of the time of this filing) alone or in combination with an immune checkpoint inhibitor (ICI) (e.g. an anti-PD-1 antibody). The symbol “a” may be used interchangeably with “anti” for terms describing an antibody (e.g. α-PD-1 antibody).

Briefly, mice bearing orthotopic EMT-6 syngeneic breast carcinoma mouse model were intravenously injected once weekly for 4 consecutive weeks with mouse ERY-MET™ (equivalent to alternately used “ERY-MET™”) at 30 U/kg or 60 U/kg alone or in combination with anti-PD-1 antibody (intraperitoneal, 10 mg/kg, twice weekly for 3 consecutive weeks) from D7 (D0 referring to injection of tumor cells). ERY-MET™ treatment was accompanied by daily oral administration of PN, which is a precursor to the MGL co-factor PLP. Mouse body weight, as well as the length and width of the tumor, were measured twice a week. Tumors from animals receiving 60 U/kg of ERY-MET™ or vehicle were collected throughout the study for metabolite measurement, immunophenotyping and/or identification of biomarkers. FIGS. 1-15 summarize the results.

Analysis of health parameters throughout the study revealed that all treatments were well tolerated by animals bearing the OT EMT-6 model. Several growth parameters were considered to evaluate the benefit of Erymethionase for improving the response to anti-PD-1 treatment. A delay in entrance in growth exponential phase was reported in case of combination and at the highest dose of Erymethionase vs single agent leading to a significant growth inhibition at D20 or D23 and an increase in survival of animals (median survival time of 23 days for anti-PD-1 or Erymethionase 60 U/kg alone vs 35 days for combination). The antitumor effects were less pronounced in case of treatment anti-PD-1 plus Erymethionase 30 U/kg. Interestingly, when EMT6 tumor cells were treated with increasing concentrations of MGL, PD-L1 expression appeared to increase (FIG. 5). Not wishing to be bound by theory, these observations could indicate that MGL may sensitize tumor cells to anti-PD-1 therapy—at least in part—by increasing PD-L1 expression levels.

Brief Conclusion. This is the first in vivo demonstration of anti-PD-1 therapy potentiation against EMT-6 TNBC cells using a methionine-depleting agent (MDA). Methioninase is on a path for first-in-human administration as single agent and in parallel optimization of regimens at the preclinical level should allow to envision a clinical evaluation of combination in several years.

### Detailed Study Design.

Test and reference substances included: Anti-PD-1 antibody (ERY-MET™: see Erytech's U.S. Pat. No. 10,046,009 B2; ref: BE0146, BioXcell; clone: RMP1-14; reactivity: mouse; isotype: Rat IgG2a; storage conditions: +4° C.); Doxorubicin (DOXO-cell®, 2 mg/mL, Cell Pharm). ERY-MET™ was prepared in AS-3/20% decomplemented BALB/C plasma, the PN working solution and Doxorubicin were prepared in 0.9% sodium chloride (NaCl), and the anti-PD-1 antibody was prepared in PBS (BE17-516F, Lonza).

Doses for the test and reference substances included the following: ERY-MET™ at 30 U/kg (dose #1) or 60 U/kg (dose #2); PN at 4.28 mg/kg; GRLR at the same maximal dose as ERY-MET™ (i.e. same volume “mL/kg”) as ERY-MET™ dose #2); Anti-PD-1 antibody at 10 mg/kg; and Doxorubicin at 5 mg/kg. As regards the routes of administration, test and reference substances were injected intravenously (IV, slow injection, also called “infusion”) into the caudal vein of mice. The recommended pH formulation for IV route is 4.5-8. The PN was administered by oral gavage (per os, PO) via a gavage tube. The recommended pH formulation for PO route is 4.5-8. Finally, the anti-PD-1 antibody was injected into the peritoneal cavity of the mice (intraperitoneally, IP). The recommended pH formulation for IP route is physiological (approximately pH 7.3-7.4.). The dose volume for test and reference substances was 10 mL/kg (i.e. for one mouse weighing 20 g, 200 μL of dosing solution was administered) and was calculated according to the most recent mouse body weight.

EMT-6 tumor cells (ATCC® CRL-2755™) were grown as a monolayer at 37° C. in a humidified atmosphere (5% CO2, 95% air). The culture medium was RPMI 1640 containing 2 mM L-glutamine (ref: BE12-702F, Lonza) supplemented with 10% fetal bovine serum (ref: P30-1506, PAN). Tumor cells were detached from the culture flask by a 5-minute treatment with trypsin-versene (ref: BE17-161E, Lonza), in Hanks' medium without calcium or magnesium (ref: BE10-543F, Lonza) and neutralized by addition of complete culture medium. The cells were counted in a hemocytometer and their viability assessed by 0.25% trypan blue exclusion assay.

One hundred twenty-two (122) healthy female BALB/c (BALB/cByJ) mice, 6-7 weeks old, were obtained from CHARLES RIVER (L'Arbresles, France). The mice were maintained in SPF health status according to the relevant standards and housed according to the following: Temperature: 22±2° C.; Humidity 55±10%; Photoperiod (12 h light/12 h dark); HEPA filtered air; 15 air exchanges per hour with no recirculation. Moreover, complete food was provided for immunocompetent rodents—R/M-H Extrudate used during acclimation period and at start of study then replaced by A04 controlled standard maintenance diet (Safe®, France) used few days before randomization and so start of treatments and until the end of the study.

Induction of EMT-6 tumors in animals. The mice were anaesthetized with Isoflurane and a 5 mm incision was made in the skin over the lateral thorax to expose mammary fat pad (MFP). About 2.5×10⁵ EMT-6 breast cells suspended in a volume of 50 μL RPMI 1640 medium were injected into the MFP tissue (right upper udder) by means of a tuberculin syringe taking care to avoid the subcutaneous space. After injection of the tumor cells, the syringe was removed and the thoracic surface was gently dabbed with a 95% ethanol-dampened cotton-swab to kill tumor cells that may leak from the injection site. The day of injection was designated D0.

The treatment started when the tumors reached a mean volume of 50-100 mm³. Eighty six (86) out of the hundred and twelve (112) mice were randomized according to their individual tumor volume into eight (8) groups each of ten (10) or thirteen (13) animals using Vivo Manager® software (Biosystemes, Couternon, France). Randomization was designated “DR”, with all treatments commencing on DR.

TABLE 1
Treatment schedule

| | No. | | | | | Treatment |
|---|---|---|---|---|---|---|
| Group | Animals | Treatment | Dose | | Route | schedule |
| 1 | 10 | Vehicle | — | | IP | TWx3 |
| 2 | 10 + 3 | GRLR | Same volume | | IV | Q7DX4 |
| | | | as ERY-MET ™ | | | |
| | | | dose #2 | | | |
| 3 | 10 | Anti-PD-1 | 10 | mg/kg | IP | TWx3 |
| 4 | 10 | ERY-MET ™ | 30 | U/kg | IV | Q7DX4 |
| | | PN | 4.28 | mg/kg | PO | Q1Dx28 |
| 5 | 10 + 3 | ERY-MET ™ | 60 | U/kg | IV | Q7DX4 |
| | | PN | 4.28 | mg/kg | PO | Q1Dx28 |
| 6 | 10 | ERY-MET ™ | 30 | U/kg | IV | Q7DX4 |
| | | PN | 4.28 | mg/kg | PO | Q1Dx28 |
| | | Anti-PD-1 | 10 | mg/kg | IP | TWx3 |
| 7 | 10 | ERY-MET ™ | 60 | U/kg | IV | Q7DX4 |
| | | PN | 4.28 | mg/kg | PO | Q1Dx28 |
| | | Anti-PD-1 | 10 | mg/kg | IP | TWx3 |
| 8 | 10 | Doxorubicin | 5 | mg/kg | IV | Q4DX4 |
| Total | 80 + 6 | | | | | |

Concomitant treatments were performed sequentially and as follows: the day of ERY-MET™ treatment, IP injection was performed before IV injection (morning) and PO administration was performed (afternoon) 6 hours after IV injection. IP and IV treatments were performed successively; and, the day without ERY-MET™ treatment, PO administration was performed before IP injection (morning).

Sample Collection. Twenty-four hours before the 1^{st} treatment and 24 hours after the last treatment, blood was collected by jugular vein puncture from all mice of groups 1-7 into blood collection tubes containing Lithium Heparin as anticoagulant. The tubes were immediately centrifuged at 1000 g for 10 minutes at +4° C. to obtain plasma. The plasma samples (1 tube per animal, 50 μL/tube) were stored in 1.5 mL propylene tubes at −80° C. until shipment (in cases where insufficient plasma was collected, the volume was adjusted to 50 μL with 0.9% NaCl, and appropriate notations were made). The maximum volume of blood that was collected was adjusted to the body weight of animals. As regards tumor collection, satellite mice from groups 2 and 5 (3 per group) were sacrificed around D15 so when tumor reach a volume of between about 500 and about 1000 mm³. Tumors were collected and cut into two parts that were weighed, snap-frozen and stored at −80° C. until analysis.

Clinical monitoring. All study data, including animal body weight measurements, tumor volume, clinical and mortality records, and treatment were scheduled and recorded on Vivo Manager® database (Biosystemes, Dijon, France). The viability and behavior were recorded every day and body weights were measured twice a week. The length and width of the tumor were measured twice a week with calipers and the volume of the tumor was estimated by the following formula: Tumor volume=(width²×length)/2. A tumor volume of 1000 mm³ is considered to be equal to 1 g. Humane endpoints were those known to the skilled artisan, including tumors exceeding 10% of normal body weight or exceeding 1500 mm³, tumors interfering with ambulation or nutrition, >8 mm ulcerated tumor, infection, bleeding, etc. Moreover, the following evaluation criteria of health were determined using Vivo Manager® software (Biosystemes, Couternon, France): individual and mean (or median) animal body weights; mean body weight change (MBWC): average weight change of treated animals in percent (weight at day B minus weight at day A divided by weight at day A). The intervals over which MBWC were calculated were chosen as a function of body weight curves and the days of body weight measurement.

Efficacy Assessment. The treatment efficacy was assessed in terms of the effects of the test substances on the tumor volumes of treated animals relative to control animals. The following evaluation criteria of antitumor efficacy were determined using Vivo Manager® (Biosystemes, Couternon, France):

1) individual and/or mean (or median) tumor volumes;

2) tumor doubling time (DT);

3) tumor growth inhibition (T/C %) defined as the ratio of the median tumor volumes of treated versus control group, calculated as: T/C %=[(median tumor volume of vehicle treated group at DX)/(median tumor volume of treated group at DX)]*100. The optimal value was the minimal T/C % ratio reflecting the maximal tumor growth inhibition achieved. The effective criteria for the T/C % ratio according to NCI standards, is 42%;

4) Relative tumor volume (RTV) curves of test and control groups were drawn. The RTV were calculated following the formula: RTV=(TV at DX)/(TV at DR), with DX: Day of measurement; DR: Day of randomization. Volume V and time to reach V. Volume V is defined as a target volume deduced from experimental data and chosen in exponential phase of tumor growth. For each tumor, the closest tumor volume to the target volume V were selected in tumor volume measurements. The value of this volume V and the time for the tumor to reach this volume were recorded. For each group, the mean of the tumor volumes V and the mean of the times to reach this volume were calculated.

Statistical Tests. All statistical analyses were performed using Vivo Manager® software (Biosystemes, Couternon, France). Statistical analysis of mean body weights, MBWC, mean tumor volumes at randomization, mean tumor volumes V, mean times to reach V and mean tumor doubling times were performed using ANOVA. Pairwise tests were performed using the Bonferroni/Dunn correction in case of significant ANOVA results. A p-value <0.05 were considered significant.

This study was repeated using 60 U/kg and 85 U/kg for further mechanistic investigation and showed a similar efficacy trend.

### Example 2—Erymethionase Potentiates Anti-PD1 Therapy in Mice Bearing Orthotopic 4T1 Tumor Cells

The aim of the study was to evaluate the antitumor activity of ERY-MET™ and PN, a precursor of MGL's cofactor that can be converted in pyridoxal-5′-phosphate by the RBCs, alone or in combination with an immune checkpoint inhibitor (anti-PD-1 antibody) in mice bearing orthotopic 4T1 tumor cells. The 4T1 model was chosen because of its TNBC-like status, its anti-PD-1 treatment resistance and its metastatic potential. The orthotopic site was chosen as it well-reflects the tumor microenvironment. Further, the 4T1 mammary carcinoma is a highly tumorigenic and invasive transplantable tumor cell line that—unlike the majority of tumor models—is capable of spontaneously metastasizing from the primary tumor to multiple distant sites including bone, brain, lymph nodes, blood, lung and liver.

Similar to the model described in Example 1, it is envisioned that the combination of ERY-MET™ and anti-PD-1 antibody therapy will have supra-additive/synergistic efficacy against the 4T1 tumors.

Unless otherwise indicated, the various methods were carried out as described in Example 1 above. Reference substances included: anti-PD-1 antibody (ref: BE0146, BioXcell; clone: RMP1-14; reactivity: mouse; isotype: Rat IgG2a; storage conditions: +4° C.); gemcitabine (200 mg, Kabi). The ERY-MET™ and PN working solutions were prepared as above, and gemcitabine was dissolved in 0.9% NaCl. ERY-MET™ was administrated at 60 U/kg or 85 U/kg corresponding to a volume of administration comprised between 2 and 8 mL/kg (depending on the most recent mouse weight). PN was administrated at 4.28 mg/kg, anti-PD-1 antibody was administrated at 10 mg/kg and gemcitabine was administrated at 100 mg/kg. Gemcitabine was administered via IV infusion, and the other substances were administered as above.

The 4T1 cell line (mouse mammary tumor, ATCC) is a 6-thioguanine resistant cell line selected from the 410.4 tumor without mutagen treatment. When injected into BALB/c mice, 4T1 spontaneously produces highly metastatic tumors that can metastasize to the lung, liver, lymph nodes and brain while the primary tumor is growing in situ. Tumor cells were grown as a monolayer at 37° C. in a humidified atmosphere (5% CO₂, 95% air). The culture medium was RPMI 1640 containing 2 mM L glutamine (ref: BE12-702F, Lonza) supplemented with 10% fetal bovine serum (ref: P30-1506, PAN), 10 mM HEPES (ref: BE17-737E, Lonza), 4.5 g/L glucose and 1 mM Na Pyruvate (ref: BE13-115E, Lonza). Tumor cells in exponential growth phase were harvested by detachment from the culture flask by a 5-minute treatment with trypsin-versene (ref: BE02-007E, Lonza), in Hanks' medium without calcium or magnesium (ref: BE10-543F, Lonza) and neutralized by addition of complete culture medium. The cells were counted in a hemocytometer and their viability was assessed by 0.25% trypan blue exclusion assay.

Animal Study. One hundred ninety-two (192) healthy female BALB/c (BALB/cByJ) mice, 6-7 weeks old, were obtained from Charles River (L'Arbresles, France). Animals were maintained substantially as described in Example 1. The mice were anaesthetized with Isoflurane and a 5 mm incision was made in the skin over the lateral thorax to expose mammary fat pad (MFP). 1×10⁵ 4 T1 breast cells suspended in a volume of 50 μL RPMI 1640 medium were injected into the MFP tissue (right upper udder) by means of a tuberculin syringe taking care to avoid the subcutaneous space. After injection, the syringe was removed, and the thoracic surface was gently dabbed with a 95% ethanol-dampened cotton-swab to kill tumor cells that may have leaked from the injection site. The skin of the mice was closed and buprenorphine was administered as deemed necessary.

The treatment was initiated when the tumors reached a mean volume of 50-100 mm³. One hundred and forty-eight (148) of the 192 mice were randomized according to their individual tumor volume into seven (7) groups of thirteen (10+3), twenty (20) or twenty-three (20+3) animals using Vivo Manager® software (Biosystemes, Couternon, France).

TABLE 2
Treatment schedule

| | No. | | | | | Treatment |
|---|---|---|---|---|---|---|
| Group | Animals | Treatment | Dose | | Route | schedule |
| 1 | 10 + 3 | Vehicle | — | | IP | Q5Dx3 |
| 2 | 20 | Gemcitabine | 100 | mg/kg | IV | Q7DX3 |
| 3 | 20 + 3 | Anti-PD-1 | 10 | mg/kg | IP | Q5Dx3 |
| 4 | 20 + 3 | ERY-MET ™ | 60 | U/kg | IV | Q7DX3 |
| | | PN | 4.28 | mg/kg | PO | Q1Dx21 |
| 5 | 20 + 3 | ERY-MET ™ | 85 | U/kg | IV | Q7DX3 |
| | | PN | 4.28 | mg/kg | PO | Q1Dx21 |
| 6 | 20 + 3 | ERY-MET ™ | 60 | U/kg | IV | Q7DX3 |
| | | PN | 4.28 | mg/kg | PO | Q1Dx21 |
| | | Anti-PD-1 | 10 | mg/kg | IP | Q5Dx3 |
| 7 | 20 + 3 | ERY-MET ™ | 85 | U/kg | IV | Q7DX3 |
| | | PN | 4.28 | mg/kg | PO | Q1Dx21 |
| | | Anti-PD-1 | 10 | mg/kg | IP | Q5Dx3 |
| TOTAL | 130 + 18 | | | | | |

As in Example 1, concomitant treatments were performed sequentially as follows: 1) on days with ERY-MET™ treatment, Anti-PD-1 IP injection was performed before ERY-MET™ IV injection (morning) and PO administration was performed 6 hours after IV injection (afternoon); 2) on days without ERY-MET™ treatment, PO administration was performed before IP injection (morning). Samples were collected similarly as above, according to the following: plasma samples (before 1st treatment: 1 tube per animal, 75 μL/tube/24 hours after 3rd treatment with ERY-MET™ and 2 hours after PN treatment: 3 tubes per animal: 2 tubes with 75 μL/tube+1 tube with remaining volume) will be stored in 1.5 mL propylene tubes at −80° C. until shipment.

Lung and tumor collections. At time of sacrifice (after 3rd treatment with ERY-MET™ and 2 hours after PN treatment), the tumor was collected and weighed. Each tumor was cut into two parts: the first half was snap-frozen and stored at −80° C., and the other half was fixed with formalin, embedded within paraffin and stored at room temperature for later analysis. In the event of the tumor size was too small to be cut in two (<300 mm³), tumors were kept as a whole and will be snap-frozen and stored at −80° C. Main mice. At D25, 10 mice per group (groups 1-7) were culled and their tumors and lungs were collected. The lungs were weighed and the number of metastases macroscopically evaluated. For each group, the 10 harvested tumors were randomized based upon their weight and separated in 2 equivalent subgroups of 5 tumors: the first subgroup of 5 tumors were snap frozen and stored at −80° C., and the other subgroup was fixed with formalin and embedded within paraffin and stored at ambient temperature for further analysis. Around D40-D45, the 10 remaining mice of groups 2-7 were culled and their tumors and lungs collected. The lung was weighed and the number of metastases macroscopically evaluated. In case of a saturating number of lungs metastases, the weight of lungs was privileged as a readout. For each group, the 10 harvested tumors were randomized on their weight and separated in 2 equivalent subgroups of 5 tumors: the first subgroup of 5 tumors was snap frozen and stored at −80° C., and the other subgroup was fixed with formalin and embedded within paraffin and stored at ambient temperature for further analysis. The length and width of the tumor were measured twice a week as in Example 1.

### Example 3—Erymethionase Potentiates Anti-PD1 Therapy Via Depletion of Adenosine in the Tumor Microenvironment (TME)

Various studies are conducted to determine whether the methioninase is potentiating the anti-PD-1 therapy via depletion of adenosine in the TME and/or down regulation of adenosine receptor on the surface of re-activated T cells. These studies are conducted to demonstrate that methionine depletion synergizes with PD-1 blocking agents in part by lowering SAM/adenosine levels in the TME. Moreover, the reduced amount of methionine may lead to a reduction in hypermethylation of DNA that would normally allow the tumor cells to escape from various immune responses.

### Example 4—Additional Studies

Various studies may be conducted in view of the presently disclosed the invention. For example, Met restriction agents (e.g. hominex2, fumagillin, orally available live bacteria harboring METase, etc.)+anti-PD1 will be evaluated using the EMT6 model described in Example. Moreover, in vivo studies will be conducted to evaluate the combination of ERY-MET™+anti-PD-1 in the B16F10 model of melanoma; and clinical trials will be conducted to evaluate the efficacy of ERY-MET™+anti-PD1 in subjects whose cancers are not (or are no longer) responding to anti-PD1 therapy. Applicants also envision testing other ICIs in combination with MET depletion approaches. Target ICI also include anti-CTLA4, and any ICI whose ability to suppress immune responses may be effectively relieved by treatment with an immune de-repressing effective amount of a MET depleting agent, including ERY-MET™ and dietary MET restriction.

### Example 5—IFN-γ in Mo-DCs Treated with α-PD1 or α-PD-L1±MGL

Mo-DCs were prepared from CD14+ cells cultured for seven days. Immature Mo-DCs were then cultured together with T cells from a separate donor in the presence of MGL (0.2 U/mL)+/−anti-PD-1 (nivolumab; 1 μg/mL) or isotype control (hIgG4) for five days. IFN-γ production was measured by ELISA. Data are presented as individual values and mean (top graph), normalized to vehicle control (middle graph) and mean of technical replicates for each individual donor (bottom graph) (n=6) for Groups 6, 7, 9, 10, 12, 14. **p<0.01, ***p<0.001 comparing anti-PD-1 treatment to hIgG4 (−/+MGL), as determined using a RM one-way ANOVA with Sidak's multiple comparison test. Dotted line represents the mean value for vehicle alone (FIG. 6).

Mo-DCs were cultured as above, this time in the presence of MGL (0.2 U/mL)+/−α-PD-L1 (atezolizumab; 1 μg/mL) or isotype control (hIgG1) for five days. IFN-γ production was measured by ELISA. Data are presented as box & whiskers with min to max (n=6) (FIG. 7). ***p<0.001 comparing anti-PD-L1 treatment to hIgG1 (with or without MGL), as determined using a repeated measures one-way ANOVA with Sidak's multiple comparison test. Therefore, MGL does not appear to impair the IFN-gamma secretion induced by α-PD-L1.

### Example 6—IFN-γ in Mo-DCs Treated with Anti-CTLA-4±MGL

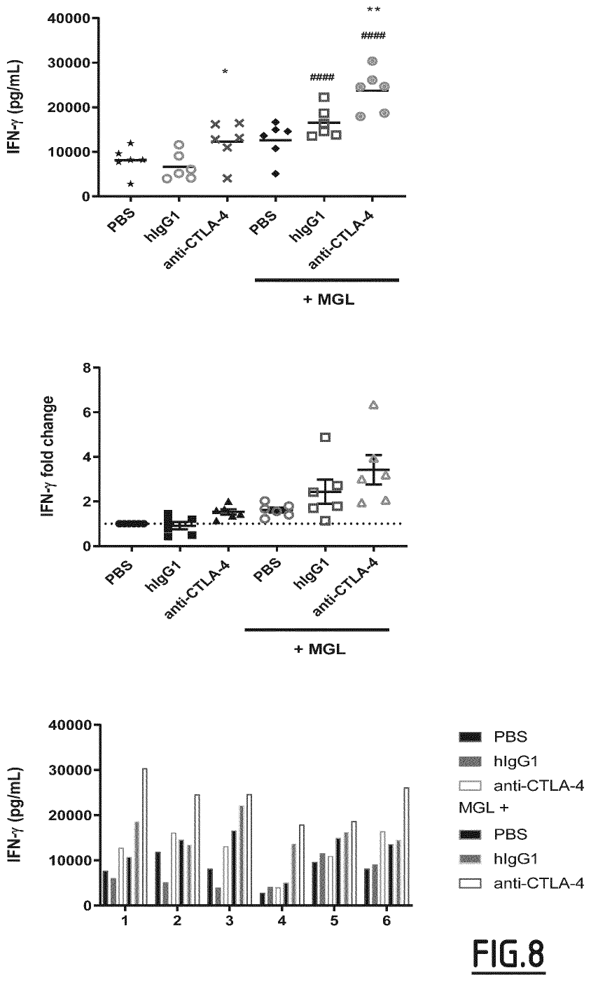

Human PBMC from two separate donors were cultured together at a 1:1 ratio+PHA (1 μg/mL) for five days in the presence of MGL (0.2 U/mL)+/−anti-CTLA-4 (Ipilimumab; 3 μg/mL) or isotype control (hIgG1). IFN-γ production was then measured by ELISA. Data are presented as individual values and mean (top graph), normalized to vehicle control (middle graph) and mean of technical replicates for each individual donor (bottom graph) (n=6). *p<0.05, **p<0.01 comparing anti-CTLA-4 treatment to hIgG1 (with or without MGL), as determined using a repeated measures one-way ANOVA with Sidak's multiple comparison test. ####p<0.0001 comparing hIgG1 or anti-CTLA-4 with MGL to hIgG1 or anti-CTLA-4 alone, as determined using a repeated measures one-way ANOVA with Sidak's multiple comparison test. Dotted line represents the mean value for vehicle alone (FIG. 8).

### Example 7—Metabolomic Data from Example 1 EMT6 Tumors

Samples produced in Example 1 were subjected to metabolomic assays and statistical analyses. Briefly, the samples were mixed with 750 μL of 50% acetonitrile in water (v/v) containing internal standards (20 μM) and homogenized by a homogenizer (1,500 rpm, 120 sec×3 times), then, the same amount of 50% acetonitrile in water (v/v) were added and centrifuged. The supernatant (400 μL) was then filtrated through 5-kDa cut-off filter (ULTRAFREE-MC-PLHCC, Human Metabolome Technologies, Yamagata, Japan) to remove macromolecules. The filtrates were centrifugally concentrated and resuspended in 50 μL of ultrapure water immediately before the metabolomic measurements (i.e. capillary electrophoresis coupled with mass spectrometry).

Turning now to the results of the metabolic analyses, Erymethionase appears to increase urea cycle metabolites, possibly to buffer Erymethionase-produced NH₃ (FIG. 9). And while ERY-MET™ does seem to elevate plasma argininosuccinate as compared to vehicle RBCs (bottom graph), the addition of α-PD-1 Abs appears to counter this effect (FIG. 9). Moreover, ERY-MET™ reduces the ratio of GSH/GSSG (FIG. 10) and, substantially reduces the plasma levels of methionine, cystathionine and (though not significantly) cysteine (a dimer form of cysteine) (FIG. 11). Further still, since cystathionine is a precursor of cysteine, and since some cancer cells are highly dependent upon extracellular cystine/cysteine, ERY-MET's ability to reduce plasma cystathionine (and possibly cysteine) likely contributes to its MOA against cancer.

As regards other analytes, ERY-MET™ increases tumor (but not plasma) 3-hydroxybutyric acid (3HB), and while the addition of α-PD-1 Abs appears to have no effect on the level of 3HB in the tumor, it does appear to increase the level of 3HB in the plasma (FIG. 12). That said, neither ERY-MET™ nor α-PD-1 Abs appear to impact 2-hydroxybutyric acid (2HB) levels in the tumor, and only ERY-MET™ appears to increase 2HB levels in the plasma (FIG. 12 bottom graphs). Furthermore, ERY-MET™ was shown to increase tumor HMG-CoA levels, and although ERY-MET™ did not significantly affect plasma acetoacetic acid levels, α-PD-1 Abs appeared to elevate plasma acetoacetic acid levels (FIG. 12, second page). Anti-PD-1 antibodies also decreased plasma lactic acid levels (FIG. 13, top) and both ERY-MET™ and α-PD-1 antibodies appear to elevate tumor lactic acid levels (FIG. 13, bottom).

Moreover, both ERY-MET™ and α-PD-1 antibodies appear to elevate plasma (but not tumor) acetamidobutanoic acid levels (FIG. 14, top graphs). Similarly, both ERY-MET™ and α-PD-1 antibodies appear to elevate tumor (but not plasma) fumarate levels, but this effect does not appear to be additive (FIG. 14, top graphs). And in a close parallel to the previous dicarboxylate, tumor malic acid levels were elevated by both ERY-MET™ and α-PD-1 antibodies, with the latter also appearing to reduce plasma malic acid levels (FIG. 14, second page). And finally, the combination of ERY-MET™ and α-PD-1 Abs significantly lowered plasma alanine levels vs. vehicle (FIG. 15).

Overall Conclusions. Taken together, the foregoing results suggest that erymethionase, and in particular ERY-MET™, may provide a novel approach to overcoming α-PD-1 resistance in various tumors. Furthermore, Applicants have demonstrated that combinations of erymethionase and ICIs outside of α-PD-1 antibodies (e.g. α-CTLA-4 antibodies) are able to produce supra-additive and/or synergistic efficacy against cancer cells. Applicants have also demonstrated that ERY-MET™ may be exerting its anti-cancer effects by modulating the levels of analytes beyond its primary substrate methionine. Notably, ERY-MET™ reduced plasma cystathionine levels, potentially revealing an important component of this drug's MOA against cancer.

### Embodiments of the Disclosure

Embodiment 1. A method for activating a suppressed (optionally tumor-infiltrating) CD8⁺ T cell to be capable of killing PD-L1 positive tumor cells in vivo in a patient suffering from a cancer comprising said tumor cells, wherein said patient's CD8⁺ T cells are being, or have been, suppressed by the combined or separate action of pathologically high levels of adenosine in the tumor microenvironment (TME) and by enhanced A2A receptor expression in said T cells, wherein said enhanced expression has been mediated, or is being mediated, by the blockade of the T cell's PD-1 pathway (optionally via the action of an αPD-1 antibody or other PD-1 pathway blocking agent), comprising the following steps:

a) administering to said patient a T cell suppressing amount of PD-1 blocking agent (optionally a α-PD-1 antibody);

b) administering to said patient a PD-1 blockade suppression-reversing amount of a methionine depletion agent (MDA); and

c) allowing a sufficient time for the MDA to reduce the level of SAM and adenosine to such an extent that a formerly suppressed T cell is now re-activated and capable of killing a PD-L1 positive tumor cell;

optionally wherein the PD-1 blocking agent is selected from Nivolumab (PD-1), Pembrolizumab (PD-1), Atezolizumab (PD-L1), Avelumab (PD-L1), Durvalumab (PD-L1), an affimer biotherapeutic inhibitor (PD-L1) (AVACTA), biosimilars thereof and combinations thereof;

optionally wherein the Pembrolizumab is Keytruda®, the Nivolumab is Opdivo®, the Cemiplimab is Libtayo®, the Atezolizumab is Tecentriq®, the Avelumab is Bavencio®, and/or the Durvalumab is Imfinzi®.

Embodiment 2. A pharmaceutical composition, kit or fixed-dose combination comprising:

(a) a methionine depletion agent (MDA); and

(b) an anti-cancer immune modulator (ACIM);

for use in the treatment of a of disease or condition in a subject or patient in need of treatment thereof;
wherein the disease or condition is not effectively treated by either the MDA or the ACIM alone; or wherein the amounts of the MDA and the ACIM are synergistically effective in treating the disease or condition; or
wherein the amount of the ACIM is sufficient to sensitize MDA-resistant cells to MDA; or
wherein the amount of the ACIM is sufficient to enable the use of a smaller amount of MDA to treat a disease or condition wherein an effective amount of the MDA would produce unacceptable toxicity in the subject or patient; or
wherein the amount of the MDA is sufficient to sensitize ACIM-resistant cells to ACIM; or
wherein the amount of the ACIM is sufficient to sensitize MDA-resistant cells to ACIM; or
wherein the amount of the MDA is sufficient to enable the use of a smaller amount of ACIM to treat a disease or condition wherein an effective amount of the ACIM would produce unacceptable toxicity in the subject or patient.

Embodiment 3. The pharmaceutical combination of Embodiment 2, wherein the MDA is a METase and the ACIM is an immune checkpoint inhibitor (ICI), and wherein the MDA and ACIM are separate entities, delivered sequentially or simultaneously, and are present in synergistically therapeutically effective amounts; optionally wherein the ICI is selected from an inhibitor of PD-1, PD-L1, CTLA4, functional equivalents thereof and combinations thereof.

Embodiment 4. The pharmaceutical combination of Embodiment 3, wherein the ICI is selected from Ipilimumab (CTLA-4), Nivolumab (PD-1), Pembrolizumab (PD-1), Atezolizumab (PD-L1), Avelumab (PD-L1), Durvalumab (PD-L1), an affimer biotherapeutic inhibitor (PD-L1) (AVACTA), biosimilars thereof and combinations thereof.

Embodiment 5. A method of treating cancer, comprising administering to a subject in need thereof synergistically effective amounts of an MDA and a ACIM.

Embodiment 6. The method of Embodiment 5, wherein the amount of the MDA would be subtherapeutic for the subject if it were not administered sequentially or simultaneously as a combination therapy with the ACIM; and/or wherein the amount of the ACIM would be subtherapeutic for the subject if it were not administered sequentially or simultaneously as a combination therapy with the MDA.

Embodiment 7. The method of Embodiment 5 or 6, wherein the amount of the MDA would be insufficient to reduce the size and/or proliferative potential of the subject's cancer were it not administered sequentially or simultaneously as a combination therapy with the ACIM; and/or wherein the amount of the ACIM would be insufficient to reduce the size and/or proliferative potential of the subject's cancer were it not administered sequentially or simultaneously as a combination therapy with the MDA.

Embodiment 8. The method of any one of Embodiments 5 to 7, wherein the cancer is acute lymphoblastic leukemia (ALL), acute myeloid leukemia (AML), pancreatic cancer, gastric cancer, colorectal cancer, prostate cancer, ovarian cancer, brain cancer, head and neck cancer or breast cancer.

Embodiment 9. The method of any one of Embodiments 5 to 8, wherein the cancer is resistant to MDA monotherapy, ACIM monotherapy or both.

Embodiment 10. The method of any one of Embodiments 5 to 9, wherein the MDA and the ACIM are sequentially administered.

Embodiment 11. The method of any one of Embodiments 5 to 10, wherein the cancer comprises a cancer-initiating stem cell.

Embodiment 12. The method any one of Embodiments 5 to 11, wherein the cancer comprises cells that are resistant to METase-mediated increases in the phosphorylation of focal adhesion kinase (FAK), activity and mRNA expression of matrix metalloproteinases MMP-2 and MMP-9, or mRNA expression of tissue inhibitor of metalloproteinase 1; or, the cells are resistant to METase-mediated decreases in urokinase plasminogen activator (uPA) and upregulation of plasminogen activator inhibitor 1 mRNA expression; and/or wherein the METase functions as a positive immune modulator.

Embodiment 13. The method of any one of Embodiments 5 to 11, wherein the cancer comprises cells that are resistant to the ACIM, but wherein sensitivity of said cells to ACIM is restored through the action of the MDA.

Embodiment 14. The method of Embodiment 13, wherein the ACIM is an anti-PD-1 antibody and the MDA is erythrocyte-encapsulated METase and the cancer comprises pancreatic, colorectal or breast cancer.

Embodiment 15. The method of Embodiment 14, wherein the cancer comprises a breast cancer.

Embodiment 16. The method of any one of Embodiments 5 to 15, wherein the ACIM and the MDA are both administered intravenously.

Embodiment 17. The method of any one of Embodiments 5 to 16, wherein the MDA METase has the sequence encoded by Gen Bank: D88554.1.

Embodiment 18. The method of any one of Embodiments 5 to 17, wherein the MDA and the ACIM are separate entities.

Embodiment 19. The method of any one of Embodiments 5 to 18, wherein the MDA is a METase encapsulated in erythrocytes (by any process, including hypotonic loading, mechanical loading, genetic expression, and any combinations thereof) and the ACIM is co-formulated with said erythrocytes.

Embodiment 20. The method of any one of Embodiments 5 to 18, wherein the ACIM is no co-formulated with the MDA, but the ACIM is co-infused into the same vessel as is the MDA.

Embodiment 21. A pharmaceutical composition, kit or fixed dose combination for use in treatment of cancer in subject in need of treatment therefor, comprising a pharmaceutically acceptable carrier and a combination of an ACIM and an MDA, wherein the combination contains a subtherapeutic dose of the ACIM and a subtherapeutic dose of the MDA, and neither the dose of the ACIM nor the dose of the MDA are or would be sufficient alone to treat the cancer.

Embodiment 22. The composition for the use of Embodiment 21, comprising at least one dose of the ACIM and at least one dose of the MDA.

Embodiment 23. The composition for the use of Embodiment 21 or 22, comprising from about 0.05 mg/kg to about 50 mg/kg bodyweight of the ACIM and from about 20 to about 100 IU/kg bodyweight of the MDA (or an amount of dietary restriction that is functionally similar to about 20 to about 100 IU/kg METase).

Embodiment 24. The composition for the use of any one of Embodiments 21 to 23, wherein the dose of the ACIM is from about 5 to about 25 mg/kg bodyweight of the subject and the dose of the MDA is about 30 to about 100 IU/kg bodyweight of the subject.

Embodiment 25. The composition for the use of any one of Embodiments 21 to 24, wherein the dose of the ACIM is from about 5 to about 20 mg/kg and the dose of the MDA is about 50 to about 100 IU/kg.

Embodiment 26. The composition for the use of any one of Embodiments 21 to 25, wherein the dose of the ACIM is from about 5 to about 15 mg/kg or about 10 mg/kg; and the dose of the MDA is about 50 to about 80 IU/kg.

Embodiment 27. The composition for the use of any one of Embodiments 21 to 26, wherein the dose of the ACIM is about 10 mg/kg and the dose of the MDA is about 60 IU/kg.

Embodiment 28. The composition for the use of any one of Embodiments 21 to 27, wherein the ACIM is an anti-PD-1 antibody and the MDA is RBC-encapsulated METase.

Embodiment 29. The composition for the use of any one of Embodiment 21 to 28, comprising from about 5 to about 15 mg/kg ACIM, optionally dissolved in suitable delivery vehicle; and about 50 to 70 IU/kg MDA.

Embodiment 30. A pharmaceutical combination comprising (i) an MDA and (ii) an ACIM and at least one pharmaceutically acceptable carrier.

Embodiment 31. The pharmaceutical combination according to Embodiment 30 for simultaneous, separate or sequential use of the components (i) and (ii).

Embodiment 32. The pharmaceutical combination according to Embodiment 30 or 31 in the form of a fixed combination.

Embodiment 33. The pharmaceutical combination according to any one of Embodiments 30 to 32 in the form or a kit of parts for the combined administration where the ACIM and the MDA may be administered independently at the same time or separately within time intervals, especially where these time intervals allow that the combination partners are jointly active.

Embodiment 34. The pharmaceutical combination according to any one of Embodiments 30 to 33, wherein the ACIM is an anti-PD-1 antibody [selected from . . . ] or is an anti-PD-1 antibody having substantially the same in vivo PK/PD profile and mechanism of action as any of the foregoing, or combinations thereof; and wherein the MDA is METase.

Embodiment 35. The pharmaceutical combination according to any one of Embodiments 30 to 34, wherein the METase is selected from an RBC-encapsulated METase and a peg-conjugated METase.

Embodiment 35. The pharmaceutical combination according to any one of Embodiments 30 to 35, further comprising a co-agent, or a pharmaceutically acceptable salt or a prodrug thereof.

Embodiment 36. The pharmaceutical combination according to any one of Embodiments 30 to 35 in the form of a co-formulated combination product.

Embodiment 37. Use of the pharmaceutical combination or combination product according to any one of Embodiments 30 to 36 for treating cancer that is or has become resistant to treatment with either the MDA or the ACIM.

Embodiment 38. A combination of (i) a METase and (ii) an anti-PD-1 antibody, for the manufacture of a medicament or a pharmaceutical product, especially a combination or combination product according to Embodiment 30, for treating cancer.

Embodiment 39. A pharmaceutical product or a commercial package comprising a combination or combination product according to Embodiment 30, in particular together with instructions for simultaneous, separate or sequential use thereof in the treatment of an MDA and an ACIM for the treatment of cancer.

Embodiment 40. A pharmaceutical combination according to Embodiment 30, for use in the treatment of cancer or as a medicine.

Embodiment 41. A method of inducing apoptosis in a tumor cell in vivo in a mammalian subject, wherein the tumor cell is resistant to treatment with an MDA, or the tumor cell that has only been rendered quiescent and/or sensitized by said MDA, comprising administering an effective amount of an MDA, administering said ACIM, and allowing sufficient time for the tumor cells to undergo apoptosis, thereby inducing the apoptosis in the tumor cell; or

the tumor cell is resistant to treatment with an ACIM, or the tumor cell that has only been rendered quiescent and/or sensitized by said ACIM, comprising administering an effective amount of an ACIM, administering said MDA, and allowing sufficient time for the tumor cells to undergo apoptosis, thereby inducing the apoptosis in the tumor cell.

Embodiment 42. The method of Embodiment 41, wherein the MDA is administered before the ACIM; or wherein the ACIM is administered before the MDA.

Embodiment 43. The method of Embodiment 41 or 42, wherein the MDA or ACIM is administered 1, 2, 3, 4, 5 or more days prior to the administration of the ACIM or MDA.

Embodiment 44. The method of any one of Embodiments 41 to 43, wherein the ACIM is administered in an amount from about 5 to about 100 mg/kg bodyweight of the subject.

Embodiment 45. The method of any one of Embodiments 41 to 44, wherein the ACIM is administered in an amount from about 10 to about 90 mg/kg.

Embodiment 46. The method of any one of Embodiments 41 to 45, wherein the ACIM is administered in an amount from about 40 to about 80 mg/kg.

Embodiment 47. The method of any one of Embodiments 41 to 46, wherein the ACIM is an anti-PD-1 antibody and the MDA is a METase.

Embodiment 48. The method of any one of Embodiments 40 to 47, wherein the ACIM is administered in an amount from about 3 to about 25 mg/kg and the METase is administered in an amount from about 10 to about 80 IU/kg.

Embodiment 49. The method of any one of Embodiments 40 to 48, wherein the ACIM is administered in an amount from about 5 to about 15 mg/kg or about 10 mg/kg; and the METase is administered in an amount from about 20 to about 70 IU/kg or about 60 IU/kg.

Embodiment 50. The method of any one of Embodiments 40 to 49, wherein the ACIM is an anti-PD-1 antibody [specific, recite amino acid sequence] and the METase is encapsulated in enucleated RBCs.

Embodiment 51. A method of treating a subject or patient suffering from cancer and previously unsuccessfully treated with an ACIM, wherein the cancer cells of the subject or patient exhibited resistance to the ACIM, comprising administering to the subject or patient an ACIM-sensitizing-effective amount of an MDA and a tumoricidal effective amount of the previously ineffective ACIM.

Embodiment 52. The method of Embodiment 51, wherein the MDA sensitizes the cancer cells to treatment with the ACIM by trapping the cells in the S/G₂ phase.

Embodiment 53. The method of Embodiment 51 or 52, wherein the ACIM is administered in an amount from about 5 to about 100 mg/kg bodyweight of the subject.

Embodiment 54. The method of Embodiment 53, wherein the ACIM is administered in an amount from about 5 to about 80 mg/kg.

Embodiment 55. The method of Embodiment 54, wherein the ACIM is administered in an amount from about 7.5 to about 50 mg/kg, or about 10 mg/kg.

Embodiment 56. The method of Embodiment 55, wherein the ACIM is an anti-PD-1 antibody and the METase is an erythrocyte-encapsulated METase.

Embodiment 57. The method of Embodiment 56, wherein the ACIM is administered in an amount from about 5 to about 15 mg/kg and the METase is administered in an amount from about 20 to about 80 IU/kg.

Embodiment 58. The method of Embodiment 57, wherein the ACIM is administered in an amount from about 7.5 to about 12.5 mg/kg and the METase is administered in an amount from about 40 to about 70 IU/kg.

Embodiment 59. The method of any one of Embodiments 51 to 58, wherein the ACIM is ibrutinib and the METase is encapsulated in enucleated erythrocytes.

Embodiment 60. The method of any one of Embodiments 56 to 59, wherein the ACIM and the METase are administered to the subject or patient in amounts that, if given separately, would not induce killing of a majority of the cancer cells.

61. The method of any one of the preceding Embodiments, wherein the MDA is a diet low in methionine.

Embodiment 62. The method of Embodiment 61, wherein the low methionine diet is begun about 14 days before or after the administration of the ACIM.

Embodiment 63. The method of Embodiment 61, wherein the low methionine diet is begun about 7 days before or after the administration of the ACIM.

Embodiment 64. The method of Embodiment 62, wherein the low methionine diet is begun about 14 days before the administration of the ACIM.

Embodiment 65. The method of Embodiment 64, wherein the low methionine diet is begun about 7 days before the administration of the ACIM.

Embodiment 66. A method of treating a cancer in a subject in need thereof, comprising administering to the subject synergistically effective amounts of:

(a) a methionine depletion agent (MDA) or methionine depletion diet (MDD); and

(b) an anti-cancer immune modulator (ACIM).

67. The method of Embodiment 66, wherein the MDA (a) comprises a METase polypeptide, optionally encapsulated in erythrocytes, optionally selected from mature red blood cells from donors, optionally including the subject, and cultured red blood cells, optionally grown from induced pluripotent stems cells, hematopoietic stems cells, and partially differentiated self-renewing erythroblast cells.

Embodiment 68. The method of Embodiment 66 or 67, wherein the METase polypeptide is a methionine gamma lyase and comprises, consists, or consists essentially of the sequence as set forth in SEQ ID NO:1 (MHGSNKLPGFATRAIHHGYDPQDHGGALVPPVYQTATFTFPTVEYGAACFAGEQAGHFYSRISNPTLNLLEARMASL EGGEAGLALASGMGAITSTLWTLLRPGDEVLLGNTLYGCTFAFLHHGIGEFGVKLRHVDMADLQALEAAMTPATRVIY FESPANPNMHMADIAGVAKIARKHGATVVVDNTYCTPYLQRPLELGADLVVHSATKYLSGHGDITAGIVVGSQALVDR IRLQGLKDMTGAVLSPHDAALLMRGIKTLNLRMDRHCANAQVLAEFLARQPQVELIHYPGLASFPQYTLARQQMSQP GGMIAFELKGGIGAGRRFMNALQLFSRAVSLGDAESLAQHPASMTHSSYTPEERAHYGISEGLVRLSVGLEDIDDLLAD VQQALKASA) (i.e. the MGL encoded by GenBank: D88554.1), or functional variants and fragments thereof which convert MET to an a-keto acid, ammonia, and a thiol (e.g. ammonia, a-Keto glutarate and methanethiol), or is a polypeptide comprising a variant of a primate cystathionine gamma-lyase, wherein the variant cystathionine gamma lyase has methionine gamma-lyase activity, a sequence at least 95% identical to SEQ ID NO:2 (MQEKDASSQGFLPHFQHFATQAIHVGQDPEQWTSRAVVPPISLSTTFKQGAPGQHSGFEYSRSGNPTRNCLEKAVA ALDGAKYCLAFASGLAATVTITHLLKAGDQIICMDDVYGGTNRYFRQVASEFGLKISFVDCSKIKLLEAAITPETKLVWIET PTNPTQKVIDIEGCAHIVHKHGDIILVVDNTFMSPYFQRPLALGADISMYSATKYMNGHSDVVMGLVSVNCESLHNRL RFLQNSLGAVPSPIDCYLCNRGLKTLHVRMEKHFKNGMAVAQFLESNPWVEKVIYPGLPSHPQHELVKRQCTGCTGM VTFYIKGTLQHAEIFLKNLKLFTLAESLGGFESLAELPAIMTHASVLKNDRDVLGISDTLIRLSVGLEDEEDLLEDLDQALKA AHPPSGSHS), and comprises amino acid substitutions at amino acid positions corresponding to positions 59, 119 and/or 339 of SEQ ID NO: 2, the native human cystathionine gamma lyase, said substitutions being i) E59V or E59N, ii) R119L and iii) E339V.

Embodiment 69. The method of Embodiment 68, wherein the METase polypeptide comprises a sequence that is at least 80%, 85%, 90%, 95%, 96%, 97%, 98%, or 99% identical to the MGL sequence encoded by D88554.1, and which converts MET to an a-keto acid, ammonia, and a thiol.

Embodiment 70. The method of any one of Embodiments 66-69, wherein the METase polypeptide is covalently bonded via an optional linker to at least one PEG molecule, is encapsulated in erythrocytes, or is bound to an albumin-binding molecule.

Embodiment 71. The method of Embodiment 70, wherein the METase is encapsulated within enucleated erythrocytes.

Embodiment 72. The method of any one of Embodiments 66-71, wherein the ACIM (b) is selected from one or more of an immune checkpoint modulatory agent, a cancer vaccine, an oncolytic virus, a cytokine, and a cell-based immunotherapies.

Embodiment 73. The method of Embodiment 72, wherein the ACIM is a polypeptide, optionally an antibody or antigen-binding fragment thereof or a ligand, or a small molecule.

Embodiment 74. The method of Embodiment 72 or 73, wherein the immune checkpoint modulatory agent comprises

(i) an antagonist of a inhibitory immune checkpoint molecule; or

(ii) an agonist of a stimulatory immune checkpoint molecule.

75. The method of Embodiment 74, wherein the ACIM specifically binds to the immune checkpoint molecule.

Embodiment 76. The method of Embodiment 73 or 74, wherein the ACIM is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD 160, Herpes Virus Entry Mediator (HVEM), and T-cell immunoreceptor with Ig and ITIM domains (TIGIT).

Embodiment 77. The method of Embodiment 74 or 75, wherein the antagonist is a PD-L1 and/or PD-L2 antagonist optionally selected from one or more of an antibody or antigen-binding fragment or small molecule that specifically binds thereto, atezolizumab, Avelumab, and durvalumab, and wherein the cancer is optionally selected from one or more of pancreatic cancer, colorectal cancer (CRC), melanoma, breast cancer (including TNBC), non-small-cell lung carcinoma (NSCLC), bladder cancer, ovarian cancer, renal cell carcinoma, glioblastoma and glioma.

Embodiment 78. The method of 74 or 75, wherein the antagonist is a PD-1 antagonist optionally selected from one or more of an antibody or antigen-binding fragment or small molecule that specifically binds thereto, optionally selected from nivolumab, pembrolizumab, and pidilizumab.

Embodiment 79. The method of Embodiment 78, wherein the PD-1 antagonist is nivolumab and the cancer is optionally selected from one or more of breast cancer (including TNBC), Hodgkin's lymphoma, melanoma, NSCLC, hepatocellular carcinoma, renal cell carcinoma, and ovarian cancer.

Embodiment 80. The method of Embodiment 76, wherein the PD-1 antagonist is pembrolizumab and the cancer is optionally selected from one or more of melanoma, breast cancer (including TNBC), NSCLC, SCLC, head and neck cancer, and urothelial cancer; or

wherein the antagonist is a CTLA-4 antagonist optionally selected from one or more of an antibody or antigen-binding fragment or small molecule that specifically binds thereto, optionally selected from ipilimumab and tremelimumab, optionally wherein the cancer is selected from one or more of breast cancer (including TNBC), melanoma, prostate cancer, lung cancer, and bladder cancer.

Embodiment 81. A method of inhibiting the growth of a tumor and/or reducing the size and/or growth rate of a tumor, comprising: contacting the tumor with an effective amount of an METase and an effective amount of one or more immune checkpoint inhibitors (IC's); optionally wherein the tumor is selected from an adrenal cancer, a bladder cancer, a bone cancer, a brain tumor, a breast cancer tumor, a cervical cancer tumor, a gastrointestinal carcinoid tumor, a stromal tumor, Kaposi sarcoma, a liver cancer tumor, a small cell lung cancer tumor, non-small cell lung cancer, a carcinoid tumor, a lymphoma tumor, a neuroblastoma, an osteosarcoma, a pancreatic cancer, a pituitary tumor, a retinoblastoma, a basal cell tumor, a squamous cell tumor, a melanoma, thyroid cancer, or a Wilms tumor.

Embodiment 82. The method of Embodiment 81, wherein the METase is comprised within an erythrocyte and the erythrocytes are suspended in a pharmaceutically acceptable carrier.

Embodiment 83. The method of Embodiment 81 or 82, wherein the ICI is selected from the group consisting of Nivolumab (OPDIVO®), Ipilimumab (YERVOY®), Pembrolizumab (KEYTRUDA®), BGB-A317, Atezolizumab, Avelumab and Durvalumab.

Embodiment 84. A method of depleting intratumoral adenosine from a tumor or a tumor microenvironment, comprising: contacting the tumor with an effective amount of a METase.

Embodiment 85. The composition, kit, combination, use or method of any one of the preceding claims, wherein the methionine depleting agent (MDA) exerts its anti-cancer efficacy and/or potentiates the efficacy of the ACIM by reducing plasma and/or tumor methionine levels and/or by:

a) sensitizing tumor cells to α-PD-1 therapy in part by increasing PD-L1 expression levels;

b) increasing plasma argininosuccinate over vehicle RBCs;

c) decreasing the ratio of GSH to GSSG in the tumor;

d) decreasing plasma cystathionine, cysteine and/or cysteine levels;

e) increasing tumor 3-hydroxybutyric acid (3HB);

f) increasing plasma 2-hydroxybutyric acid (2HB);

g) increasing tumor HMG-CoA levels;

h) increasing lactic acid levels in the tumor;

i) increasing plasma acetamidobutanoic acid levels;

j) increasing tumor fumarate levels;

k) increasing tumor malic acid levels; and/or

l) decreasing plasma alanine levels

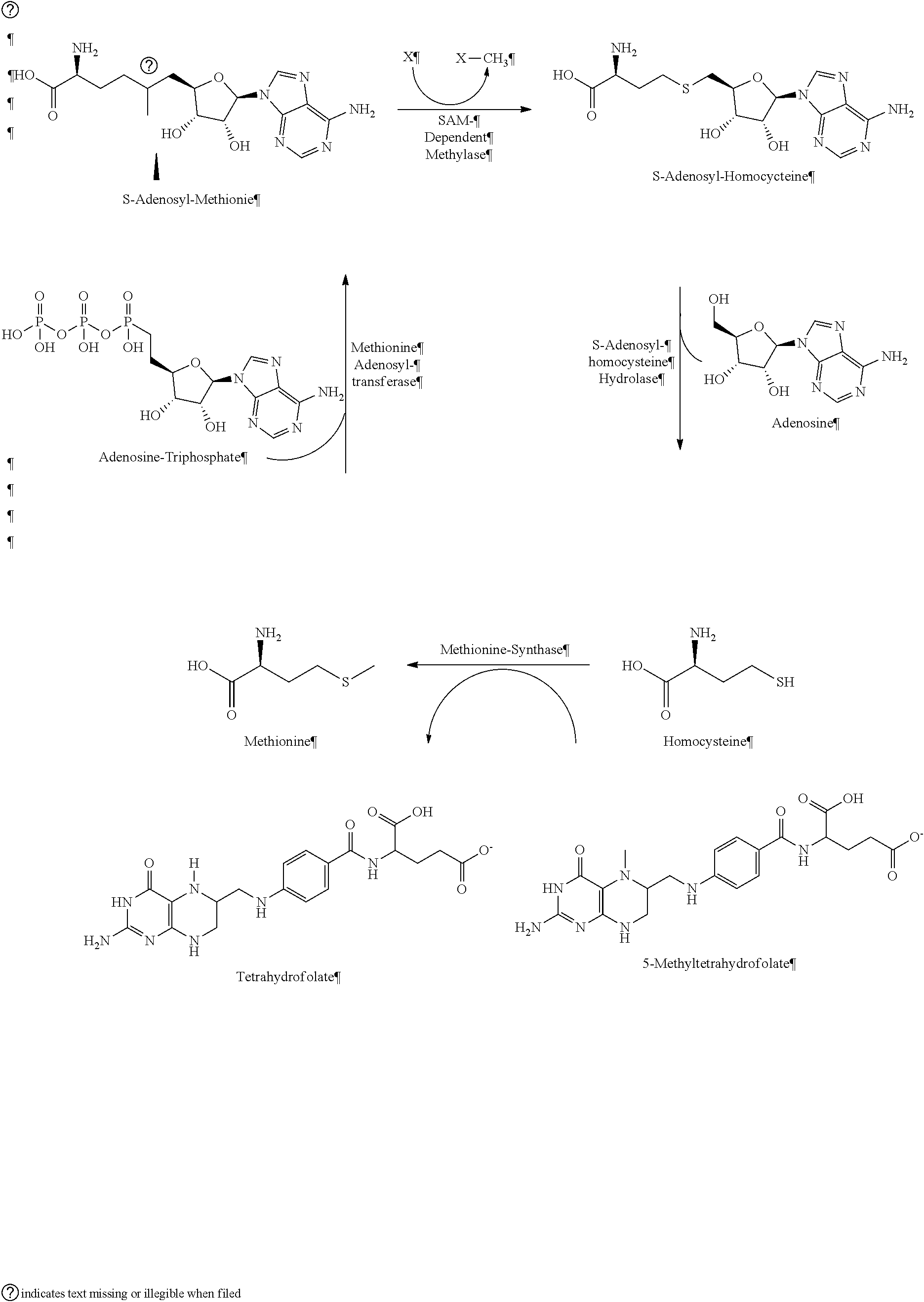

1. A pharmaceutical composition, kit or fixed-dose combination comprising:
(a) a methionine depletion agent (MDA); and
(b) an anti-cancer immune modulator (ACIM);
for use in the treatment of a disease or condition in a subject or patient in need of treatment thereof;
wherein the disease or condition is not effectively treated by either the MDA or the ACIM alone; or
wherein the amounts of the MDA and the ACIM are synergistically effective in treating the disease or condition; or
wherein the amount of the ACIM is sufficient to sensitize MDA-resistant cells to MDA; or
wherein the amount of the ACIM is sufficient to enable the use of a smaller amount of MDA to treat a disease or condition wherein an effective amount of the MDA would produce unacceptable toxicity in the subject or patient; or
wherein the amount of the MDA is sufficient to sensitize ACIM-resistant cells to ACIM; or
wherein the amount of the ACIM is sufficient to sensitize MDA-resistant cells to ACIM; or
wherein the amount of the MDA is sufficient to enable the use of a smaller amount of ACIM to treat a disease or condition wherein an effective amount of the ACIM would produce unacceptable toxicity in the subject or patient.

2. The pharmaceutical composition, kit or fixed-dose combination of claim 1, wherein the ACIM is a PD-1 blocking agent is selected from Nivolumab (PD-1), Pembrolizumab (PD-1), Atezolizumab (PD-L1), Avelumab (PD-L1), Durvalumab (PD-L1), an affimer biotherapeutic inhibitor (PD-L1) (AVACTA), biosimilars thereof and combinations thereof; preferably wherein the Pembrolizumab is Keytruda®, the Nivolumab is Opdivo®, the Cemiplimab is Libtayo®, the Atezolizumab is Tecentriq®, the Avelumab is Bavencio®, and/or the Durvalumab is Imfinzi®.

3. The pharmaceutical composition, kit or fixed-dose combination of claim 1, wherein the MDA is a METase and the ACIM is an immune checkpoint inhibitor (ICI), and wherein the MDA and ACIM are separate entities, delivered sequentially or simultaneously, and are present in synergistically therapeutically effective amounts; optionally wherein the ICI is selected from an inhibitor of PD-1, PD-L1, CTLA4, functional equivalents thereof and combinations thereof.

4. The pharmaceutical composition, kit or fixed-dose combination of claim 1, wherein the ICI is selected from Ipilimumab (CTLA-4), Nivolumab (PD-1), Pembrolizumab (PD-1), Atezolizumab (PD-L1), Avelumab (PD-L1), Durvalumab (PD-L1), an affimer biotherapeutic inhibitor (PD-L1) (AVACTA), biosimilars thereof and combinations thereof.

5. Use of the composition of claim 1 for treating cancer, wherein the MDA and ACIM are present in synergistically effective amounts.

6. The use of claim 5, wherein the amount of the MDA would be subtherapeutic for the subject if it were not administered sequentially or simultaneously as a combination therapy with the ACIM; and/or wherein the amount of the ACIM would be subtherapeutic for the subject if it were not administered sequentially or simultaneously as a combination therapy with the MDA.

7. The use of claim 5, wherein the amount of the MDA would be insufficient to reduce the size and/or proliferative potential of the subject's cancer were it not administered sequentially or simultaneously as a combination therapy with the ACIM; and/or wherein the amount of the ACIM would be insufficient to reduce the size and/or proliferative potential of the subject's cancer were it not administered sequentially or simultaneously as a combination therapy with the MDA.

8. The use of claim 5, wherein the cancer is acute lymphoblastic leukemia (ALL), acute myeloid leukemia (AML), pancreatic cancer, gastric cancer, colorectal cancer, prostate cancer, ovarian cancer, brain cancer, head and neck cancer or breast cancer.

9. The use of claim 5, wherein the cancer is resistant to MDA monotherapy, ACIM monotherapy or both.

10. The use of claim 5, wherein the MDA and the ACIM are sequentially administered.

11. The use of claim 5, wherein the cancer comprises a cancer-initiating stem cell.

12. The use of claim 5, wherein the cancer comprises cells that are resistant to METase-mediated increases in the phosphorylation of focal adhesion kinase (FAK), activity and mRNA expression of matrix metalloproteinases MMP-2 and MMP-9, or mRNA expression of tissue inhibitor of metalloproteinase 1; or, the cells are resistant to METase-mediated decreases in urokinase plasminogen activator (uPA) and upregulation of plasminogen activator inhibitor 1 mRNA expression; and/or wherein the METase functions as a positive immune modulator.

13. The use of claim 5, wherein the cancer comprises cells that are resistant to the ACIM, but wherein sensitivity of said cells to ACIM is restored through the action of the MDA.

14. The use of claim 13, wherein the ACIM is an anti-PD-1 antibody and the MDA is erythrocyte-encapsulated METase and the cancer comprises pancreatic, colorectal or breast cancer.

15. The use of claim 14, wherein the cancer comprises a breast cancer.

16. The use of claim 5, wherein the ACIM and the MDA are both administered intravenously.

17. The use of claim 5, wherein the MDA METase has the sequence encoded by Gen Bank: D88554.1 or has the sequence as set forth in SEQ ID NO: 1 or 2.

18. The use of claim 5, wherein the MDA and the ACIM are separate entities.

19. The use of claim 5, wherein the MDA is a METase encapsulated in erythrocytes (by any process, including hypotonic loading, mechanical loading, genetic expression, and any combinations thereof) and the ACIM is co-formulated with said erythrocytes.

20. The use of claim 5, wherein the ACIM is no co-formulated with the MDA, but the ACIM is co-infused into the same vessel as is the MDA.

21. A pharmaceutical composition, kit or fixed dose combination for use in treatment of cancer in subject in need of treatment therefor, comprising a pharmaceutically acceptable carrier and a combination of an ACIM and an MDA, wherein the combination contains a subtherapeutic dose of the ACIM and a subtherapeutic dose of the MDA, and neither the dose of the ACIM nor the dose of the MDA are or would be sufficient alone to treat the cancer.

22. The composition for the use of claim 21, comprising at least one dose of the ACIM and at least one dose of the MDA.

23. The composition for the use of claim 21, comprising from about 0.05 mg/kg to about 50 mg/kg bodyweight of the ACIM and from about 20 to about 100 IU/kg bodyweight of the MDA (or an amount of dietary restriction that is functionally similar to about 20 to about 100 IU/kg METase).

24. The composition for the use of claim 21, wherein the dose of the ACIM is from about 5 to about 25 mg/kg bodyweight of the subject and the dose of the MDA is about 30 to about 100 IU/kg bodyweight of the subject.

25. The composition of claim 1, wherein the MDA exerts its anti-cancer efficacy and/or potentiates the efficacy of the ACIM by reducing plasma and/or tumor methionine levels and/or by:
a) sensitizing tumor cells to α-PD-1 therapy in part by increasing PD-L1 expression levels;
b) increasing plasma argininosuccinate over vehicle RBCs;
c) decreasing the ratio of GSH to GSSG in the tumor;
d) decreasing plasma cystathionine, cysteine and/or cysteine levels;
e) increasing tumor 3-hydroxybutyric acid (3HB);
f) increasing plasma 2-hydroxybutyric acid (2HB);
g) increasing tumor HMG-CoA levels;
h) increasing lactic acid levels in the tumor;
i) increasing plasma acetamidobutanoic acid levels;
j) increasing tumor fumarate levels;
k) increasing tumor malic acid levels; and/or
l) decreasing plasma alanine levels.

26. The composition or use of claim 21, wherein the MDA exerts its anti-cancer efficacy and/or potentiates the efficacy of the ACIM by reducing plasma and/or tumor methionine levels and/or by:
a) sensitizing tumor cells to α-PD-1 therapy in part by increasing PD-L1 expression levels;
b) increasing plasma argininosuccinate over vehicle RBCs;
c) decreasing the ratio of GSH to GSSG in the tumor;
d) decreasing plasma cystathionine, cysteine and/or cysteine levels;
e) increasing tumor 3-hydroxybutyric acid (3HB);
f) increasing plasma 2-hydroxybutyric acid (2HB);
g) increasing tumor HMG-CoA levels;
h) increasing lactic acid levels in the tumor;
i) increasing plasma acetamidobutanoic acid levels;
j) increasing tumor fumarate levels;
k) increasing tumor malic acid levels; and/or
l) decreasing plasma alanine levels.


SEQUENCE LISTING (2 sequences)

SEQ ID NO: 1 - protein, 398 aa, Artificial Sequence
  Polypeptide encoding P. putida methionine gamma lyase (MGL) (GenBank  D88554.1)
MHGSNKLPGFATRAIHHGYDPQDHGGALVPPVYQTATFTFPTVEYGAACFAGEQAGHFYSRISNPTLNLLEARMASLEGGEAGLALASGMGAITSTLWTLLRPGDEVLLGNTLYGCTFAFLHHGIGEFGVKLRHVDMADLQALEAAMTPATRVIYFESPANPNMHMADIAGVAKIARKHGATVVVDNTYCTPYLQRPLELGADLVVHSATKYLSGHGDITAGIVVGSQALVDRIRLQGLKDMTGAVLSPHDAALLMRGIKTLNLRMDRHCANAQVLAEFLARQPQVELIHYPGLASFPQYTLARQQMSQPGGMIAFELKGGIGAGRRFMNALQLFSRAVSLGDAESLAQHPASMTHSSYTPEERAHYGISEGLVRLSVGLEDIDDLLADVQQALKASA

SEQ ID NO: 2 - protein, 405 aa, Artificial Sequence
  Polypeptide encoding cysteine gamma lyase (CGL)
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS